(19) United States
(12) Patent Application Publication
(10) Pub. No.: US 2007/0264355 A1
(43) Pub. Date: Nov. 15, 2007

(54) BINARY COMPOSITIONS AND METHODS FOR STERILIZATION

(75) Inventors: Robert C. Allen, Omaha, NE (US); Suzan Woodhead, Athens, OH (US); Sophie Becquerelle, Sebastopol, CA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Binary, LLC, Little Rock, AR (US)

(21) Appl. No.: 11/611,087

(22) Filed: Dec. 14, 2006

Related U.S. Application Data
(60) Provisional application No. 60/750,764, filed on Dec. 14, 2005.

(51) Int. Cl. A61K 33/40; A61K 33/14

(52) U.S. Cl. 424/615; 424/613; 424/616; 424/665; 424/722

(57) ABSTRACT

The present invention relates to binary methods and compositions comprising hypohalite (preferably hypochlorite) and peroxide (preferably hydrogen peroxide) directed to the killing of pathogenic microbes such as parasites, bacteria, fungi, yeast, and prions, the oxidation of toxins, and the preparation of potable water. The binary methods and compositions extend the microbicidal potency of conventional hypochlorite by providing additional singlet molecular oxygen generated in situ, and offer more control over reactive chlorination exposure than hypochlorite alone. This combination is a highly effective disinfecting and decontaminating agent, capable of disinfection, detoxification, or deactivation of biological contamination and many chemical toxins, facilitating the sterilizing of surfaces and solutions, and the production of potable water.

### CROSS REFERENCES TO RELATED APPLICATIONS

This application claims the benefit of U.S. Provisional Application No. 60/750,764, filed on Dec. 14, 2005, the disclosure of which is incorporated by reference.

### FIELD OF THE INVENTION

The present invention relates to binary methods and compositions comprising hypohalite (preferably hypochlorite) and peroxide (preferably hydrogen peroxide) directed to the killing of pathogenic microbes such as parasites, bacteria, fungi, yeast, and prions, the oxidation of toxins, and the preparation of potable water. The binary methods and compositions extend the microbicidal potency of conventional hypochlorite by providing additional singlet molecular oxygen generated in situ, and offer more control over reactive chlorination exposure than hypochlorite alone. This combination is a highly effective disinfecting and decontaminating agent, capable of disinfection, detoxification, or deactivation of biological contamination and many chemical toxins, facilitating the sterilizing of surfaces and solutions, and the production of potable water.

### BACKGROUND OF THE INVENTION

Antiseptics and disinfectants are used extensively in health care and food service settings and in general consumer markets to prevent the growth and transmission of infectious agents, particularly bacteria and viruses. A wide variety of natural and synthetic antimicrobial chemical agents, or biocides, are used in connection with these products, which are often used in combination, to enhance their activity on multiple intracellular or extracellular targets. Some agents, such as alcohols, have a broad spectrum of activity against microorganisms, while others, such as antibiotics, typically have a much narrower spectrum of activity, effective against related types of bacteria. Biocides fall into several classes, which include alcohols, aldehydes, anilides, biguanides, bisphenols, diamidines, halogen releasing agents, halophenols, heavy metal derivatives, peroxygens, phenols and cresols, quaternary ammonium compounds, and vapor phase agents. The major features of many biocides, including their chemical structures, mechanism of action, and use for antisepsis, cleaning, deodorization, disinfection, preservation, or sterilization has been reviewed (McDonnell, G., and Russell, A. D. (1999) Microbiol. Rev. 12(1): 147-179).

Different types of microorganisms or infectious agents vary in their response to biocides. In ascending order of resistance to antiseptics and disinfectants, they include lipid enveloped viruses, Gram-positive bacteria, large non-enveloped viruses, fungi, trophozoites, small non-enveloped viruses, cysts, mycobacteria, spores, coccidia, and prions. Intrinsic mechanisms of resistance include impermeability, enzymatic inactivation, and efflux, while acquired mechanisms include transmission of heritable genetic material and mutation. Impermeability, for example, can be mediated by waxy cell walls, extracellular polysaccharide layers, and peptidoglycan coats, for mycobacteria, Gram-positive bacteria, and spores, respectively.

Oxidizing agents such as hydrogen peroxide (H₂O₂), peracetic acid (CH₃COOOH), and hypochlorite (bleach) are often used as biocides for disinfection, sterilization, and antisepsis. Hypochlorite is considered to be the most effective and efficient agent for biological and chemical decontamination, although its high level of corrosiveness and inherent toxicity is considered to be a significant disadvantage. Concentration and contact time are primary factors in determining the efficacy and corrosiveness of hypochlorite solutions. H₂O₂ which is available as a liquid in concentrations ranging from 3 to 90%, is also effective against a broad range of viruses, bacteria, yeasts, and bacterial spores. These compounds are believed to act by producing hydroxyl free radicals (*OH) which attack essential cell components, by disrupting sulfhydryl (—SH) and sulfur (S—S) bonds (McDonnell and Russell (1999), supra). A brief history of the development and use of these and related oxidizing agents follows.

Hypochlorite

The French chemist Berthollet described the disinfecting and bleaching properties of a solution prepared from aqueous alkali and chlorine in 1788, and in 1792, a potassium-based preparation of similar composition, eau de Javel, was sold commercially as a disinfectant. In 1820 Labarraque prepared a solution from aqueous sodium carbonate and chlorine. This liqueur de Labarraque was well known for its disinfectant and deodorizer qualities. In 1846, Semmelweis used chloride of lime (calcium hypochlorite) solution, to successfully control the spread of puerperal sepsis in Austria. In 1881, Koch also reported on the bactericidal action of hypochlorite.

Hydrogen Peroxide

In 1818, French chemist Thenard synthesized hydrogen peroxide (H₂O₂) by reacting dilute acid with barium dioxide, to yield a 3 to 4% solution of H₂O₂. The disinfectant properties of H₂O₂ were well recognized by the mid nineteenth century and were advocated for use in rendering water and milk safe, for the disinfection of sewage, with applications including medicine, surgery, dentistry, and hair-dressing (Heinemann, 1913, J.A.M.A. 60: 1603-6). The antiseptic capacity of these peroxides was relatively poor, however, compared to hypochlorites.

Photooxidation

The antiseptic action of dyes was well known before World War I (WWI). In 1900, Raab reported that the dye acridine killed living cells (paramecia), but only in the presence of light (Z. Biol. 39: 524 et seq.). In 1905, Jodlbauer and von Tappeiner demonstrated that O₂ was also required for the dye-sensitized photo-killing of bacteria (Deut. Arch. Klin. Med. 82: 520-46). Dye-sensitized, O₂-dependent photooxidation and photooxygenation reactivity is commonly referred to as photodynamic activity (Blum, 1941, Photodynamic Action and Diseases Caused by Light, Reinhold, N.Y.). Dyes, such as flavine and brilliant green, are effective as antiseptic agents, even when employed at relatively high dilutions in serous medium. Unfortunately, their potent antimicrobial action is offset by damage to or killing of host cells, including leukocytes (Fleming, 1919, Brit. J. Surg. 7: 99-129).

Wound Antisepsis

The potency of hypochlorite-based antiseptics (Andrewes and Orton, 1904, Zentrabl. Bakteriol. (Orig. A) 35: 811-6) was firmly established during WWI. By the end of the war, Eusol (Smith et al., 1915, Brit. Med. J. 2: 129-36) and Dakin's solution (Dakin, 1915, Brit. Med. J. 2: 318-20) had replaced the preferred antiseptics, carbolic acid and iodine.

Alexander Fleming described two schools of thought regarding the treatment of wounds in his Hunterian lecture, “The Action of Chemical and Physiological Antiseptics in a Septic Wound” (1919, Brit. J. Surg. 7: 99-129): (1) the physiological school directed “their efforts to aiding the natural protective agencies of the body against infection”, and (2) the antiseptic school which directed their efforts to killing wound microbes with chemical agents.

The physiologic school maintained that the greatest protection against infection was obtained by aiding the physiological agencies: (1) blood and humoral defense mechanisms, and (2) phagocytic leukocytes. Leukocytes were known to enter into the wound, ultimately forming the cellular elements of pus. Fleming noted that the phagocytic leukocytes of “fresh pus” exert potent antimicrobial effect. “Stale pus” (from an unopened anaerobic furuncle) and heat-treated or antiseptic-treated “fresh pus”, however, lack microbe killing capacity.

Nonspecific Nature of Antiseptic Treatment

Oxidative agents such as hypochlorite can exert potent microbicidal action, but their reactivity is non-specific. Germicidal action can be competitively inhibited by reaction with the organic matter present in the fluid or on the surface to be sterilized. Disinfection is a chemical reaction “in which the reactive agent acts not only on bacteria but upon the media in which they are found” (Dakin, 1915, Brit. Med. J. 2: 809-10).

Fleming noted, “A consideration of the leucocidal property of antiseptics will show us that certain antiseptics are suitable for washing of a wound, while others are bad. If we desire, therefore, an antiseptic solution with which to wash out a wound, we should choose one which loses its anti-leucocytic power rapidly and which exercises its antiseptic action very quickly. We then have the washing effect of the fluid without doing much damage to the wound. One great advantage of eusol and Dakin's solution is that they disappear as active chemical agents in a few minutes and do not have any lasting deleterious effect on the leukocytes” (Fleming, 1919, Brit. J. Surg. 7: 99-129). Eusol and Dakin's solution are both hypochlorite-based solutions. As such, an ideal agent exerts rapid and potent microbicidal action that can be terminated before damaging host biological tissues or cells.

Mechanism of Hypochlorite Action

The microbicidal action of hypochlorite was initially thought to be dependent on nascent oxygen liberated as a product of hypochlorous acid autoprotolysis, and that this liberated oxygen was responsible for microbe killing. Dakin, however, challenged this view “It has been repeatedly stated that the antiseptic action of hypochlorous acid was due to the liberation of oxygen. I have been unable to find any evidence to support this statement.” He went on to propose a more direct chlorination mechanism. “It appears that when hypochlorous acid and hypochlorites act upon organic matter of bacterial or other origin some of the (NH) groups of the proteins are converted into (NCl) groups. The products thus formed, belonging to the group of chloramines, I have found to possess approximately the same antiseptic action as the original hypochlorite, and it appears more probable that the antiseptic action of the hypochlorites is conditioned by the formation of these chloramines rather than by any decomposition with liberation of oxygen” (Dakin, 1915, Brit. Med. J. 2: 318-20). “Oxygen from sources other than chlorine does not kill bacteria as readily as does the amount of chlorine theoretically necessary to yield an equivalent amount of nascent oxygen” (Mercer and Somers, 1957, Adv. Food Res. 7: 129-60).

Dakin's mechanism of direct chlorine microbicidal action persists to the present, although it has been challenged. “Experimental proof is lacking also for other hypotheses advanced to explain the bactericidal action of chlorine. These include suggestions that bacterial proteins are precipitated by chlorine; that cell membranes are altered by chlorine to allow diffusion of cell contents; and that cell membranes are mechanically disrupted by chlorine” (Mercer and Somers, 1957, Adv. Food Res. 7: 129-60). Chlorine-binding to bacteria is remarkably low at pH 6.5 and is doubled by raising the pH to 8.2 (Friberg, 1956, Acta Pathol. Microbiol. Scand. 38: 135-44). The bactericidal and virucidal capacity of hypochlorite, however, is increased by acidity (Butterfield et al., 1943, Publ. Health Reports 58: 1837-66; Friberg and Hammarstrom, 1956, Acta Pathol. Microbiol. Scand. 38: 127-34).

Chloramines

Organic chloramine preparations, such as chloramine-T, have long been used as antiseptic agents. Contradicting Dakin's position, the microbicidal action of chloramines is believed to result in whole or in large part from the hypochlorous acid formed from chloramine hydrolysis (Leech, 1923, J. Am. Pharm. Assoc. 12: 592-602). Chloramine bactericidal action “may be due in whole or in part to the hypochlorous acid formed in accordance with the hydrolysis and ionization equilibria” (Marks et al., 1945, J. Bacteriol. 49: 299-305).

Hypochlorite exerts a bactericidal action at concentrations of 0.2 to 2.0 ppm (4 to 40 nmol per ml). This high potency suggests that germicidal action results from the inhibition of an essential enzyme or enzymes (Green, 1941, Adv. Enzymol. 1: 177-98). Hypochlorous acid has been reported to inhibit various sulfhydryl enzymes resulting in bacterial killing (Knox et al., 1948, J. Bacteriol. 55: 451-8).

Peroxide as an Oxidizing Agent

“H₂O₂ in spite of its high oxidation-reduction potential is as sluggish an oxidizing agent as molecular oxygen and in fact a large number of oxidations attributed to this substance have been found, on careful examination, to be due to free radical formation which occurs on addition of catalytic amounts of Fe⁺⁺ or Cu⁺” (Guzman-Barron et al., 1952, Arch. Biochem. Biophys. 41: 188-202). This is the consensus conclusion of several studies (Yoshpe-Purer and Eylan, 1968, Health Lab. Sci. 5: 233-8; Miller, 1969, J. Bacteriol. 98: 949-55). More recently, the use of peroxide in combination with hypochlorite has been suggested. See, for example, U.S. Pat. No. 6,866,870, which discloses compositions comprising hypochlorite and peroxide, prepared by the addition of peroxide to hypochlorite, wherein the weight ratio of hypochlorite to peroxide is in the range of 10:1 to 100:1, and preferably being closer to 10:1.

Photodynamic Action

Photodynamic action results when a dye (a singlet multiplicity sensitizer molecule, 1Dye), absorbs a photon (hν) and is promoted to its singlet excited state (¹Dye*). If 1Dye* decays back to its 1Dye ground state by photon emission, fluorescence is observed without photodynamic action. To serve as a photodynamic sensitizer the 1Dye* must undergo intersystem crossing (ISC, a change in spin multiplicity), to yield the metastable triplet excited state of the dye (³Dye*) (Gollnick, 1968, Advan. Photochem. 6: 1-122):
¹Dye+hν→¹Dye*-ISC→³Dye* (1)

The ³Dye* state is relatively long-lived, and as such, can participate in chemical reaction with other molecules. Photodynamic reactions can be divided into two main classes depending on the reactivity of ³Dye* (Schenck and Koch, 1960, Z. Electrochem. 64: 170-7). In Type I reactions the excited triplet sensitizer is said to undergo direct redox transfer with another substrate molecule (¹SubH). Sensitizers for Type I reactions typically are readily oxidized or reduced.
³Dye*+¹SubH→²Dye+²Sub (2)

In equation (2), the triplet sensitizer serves as a univalent oxidant and is univalently reduced to its doublet state (²Dye), and the singlet multiplicity substrate (¹SubH) is oxidized to a radical doublet multiplicity (²Sub) state. The 2Dye product can react with ground state triplet multiplicity molecular oxygen (³O₂), to yield the doublet multiplicity hydrodioxylic acid radical (²O₂H) or its conjugate base the superoxide anion (²O₂⁻) and thus regenerate the singlet ground state of the dye (²Dye):
²Dye*+³O₂→¹Dye+²O₂H (or ²O₂⁻) (3)

Under acid to neutral conditions the oxygen products of reaction (3) undergo doublet-doublet (radical-radical) annihilation to yield H₂O₂:
²O₂H+²O₂⁻+H⁺→¹H₂O₂+¹O₂* (4)

If the reaction is by direct annihilation (proceeding through a singlet surface), spin conservation will be maintained and electronically excited singlet molecular oxygen (¹O₂*) will be produced (Khan, 1970, Science 168: 476-477).

In Type II reactions, the excited triplet sensitizer (³Dye*) undergo direct spin-allowed triplet-triplet annihilation with triplet ground state molecular oxygen (³O₂). Triplet-triplet annihilation proceeding through a singlet surface will yield the singlet ground state dye (¹Dye) and electronically excited singlet molecular oxygen (¹O₂*) as products (Kautsky, 1939, Trans. Faraday Soc. 35: 216-219):
³Dye*+³O₂→¹Dye+¹O₂* (5)

Reaction (5) is the most common Type II pathway. However, reaction of 3Dye* with 3O₂ can also proceed through a doublet surface, yielding doublet products:
³Dye*+³O₂→²Dye+²O₂H (6)

Doublet-doublet radical annihilation proceeding through a singlet surface will yield 1H₂O₂ as described by reaction (4).

In considering these reaction pathways it should be appreciated that reaction (5) is favored over reaction (6) by over two orders of magnitude (Kasche and Lindqvist, 1965, Photochem. Photobiol. 4: 923-33).

Singlet Molecular Oxygen

Singlet molecular oxygen (¹O₂*) is a potent electrophilic oxygenating agent. It can inhibit enzymes by oxidizing amino acids essential to catalytic activity. The rate constants (kᵣ, in M⁻¹ sec⁻¹) for the reaction of 1O₂* with tryptophan, histidine, and methionine range from 2×10⁷ to 9×10⁷ (Matheson and Lee, 1979, Photochem. Photobiol. 29: 879-81; Kraljic and Sharpatyi, 1978, Photochem. Photobiol. 28: 583-6). If generated in close proximity to a target microbe, 1O₂* can inhibit the enzymes required for microbe metabolism. Unsaturated lipids, nucleic acids and other electron dense biological molecules are susceptible to 1O₂* electrophilic attack.

An ideal sterilizing agent should exert potent reactivity against a broad range of pathogenic microbes, including parasites, bacteria, fungi, yeast, viruses, and prions. It should also possess detoxifying and deodorizing qualities. While hypochlorite is a potent microbicidal agent, its usefulness is compromised by its relatively nonspecific reactivity and corrosive properties. Hypochlorite chemical damage is not limited to the target microbe, and the duration and cessation of hypochlorite reactivity are also difficult to control.

Therefore, there exists a need for efficient and cost-effective methods and compositions for disinfecting and/or sterilizing human or animal subjects, materials, or devices, which is effective in solution and on surfaces against a wide variety of bacteria, fungi, yeasts, viruses, and prions, and is tolerated by the user, does not damage devices, and is designed for ease and convenience of storage and use. Ideally, such methods and compositions should be fast acting with minimal host toxicity and maximal germicidal action. The compositions should be inexpensive, easy to prepare and deliver, should not damage the subject, material, or device treated, and should not cause damage to host tissue on contact. Depending upon the strength of composition and the time interval of exposure, the compositions should produce antisepsis, disinfection, or sterilization.

### SUMMARY OF THE INVENTION

A binary chemical system for rapid, potent germicidal action is disclosed. The present invention provides methods of decontaminating a surface or liquid target comprising contacting the target with a first composition comprising hypohalite, preferably hypochlorite, for a first treatment time, and then contacting the target with a second composition comprising a sufficient amount of peroxide, preferably hydrogen peroxide, to react with substantially all of the hypohalite and chloramines products in the first composition for a second treatment time.

A second aspect of the invention provides kits for decontaminating a surface or a liquid target comprising a first container containing a first composition comprising hypohalite and a second container containing a second composition that comprises peroxide. In other embodiments, the present invention also provides for kits wherein the first composition, the second composition, or both first and second compositions, further comprise one or more surfactants, detergents, co-solvents, gelling agents, thixotropic agents, viscosity enhancing agents, or detection agents.

The first component (Phase I) of the binary system is hypochlorite at a concentration sufficient to produce rapid germicidal action when applied to a surface or added to a liquid. The germicidal action of Phase 1 of the invention can be represented by reaction (7):
OCl⁻_{(excess)}+Microbe→Microbe_{(dead)}+OCl⁻_{(residual)}+chloramines (7)

The second component (Phase II) of the binary system is hydrogen peroxide at and concentration sufficient to react with residual hypochlorite or its chloramines reaction products to yield singlet molecular oxygen (¹O₂*), as shown by reaction (8):
OCl⁻_{(residual)}+chloramines+H₂O₂ →¹O₂*+Cl⁻ (8)
Reaction with chloramines is expected to be slow compared to reaction with hypochlorite.

¹O₂* is a relatively short lived but potent electrophilic reactant capable of directly dioxygenating (i.e., combusting) and killing microbes. This action can be represented by reaction (9):
Microbe_{(remaining)}+¹O₂*→Microbe_{(dead)} (9)

Reactions (8) and (9) are rapid. Adding H₂O₂ causes the cessation of chlorination activity and the initiation of short-lived, but potent, singlet oxidation activity. The single multiplicity of 1O₂* allows direct reactivity with the singlet organic molecules of biological systems. 1O₂* is metastable with an aqueous reactive lifetime in the microsecond range, and as such, its reactive radius is less than 0.2 micron (micrometer) from its point of origin.

As described above in reaction (8), reaction of OCl⁻ with H₂O₂, both singlet multiplicity reactants, proceeds through a singlet multiplicity surface to yield 1O₂* and H₂O₂, all singlet multiplicity products (Kasha and Khan, 1970, Ann. N.Y. Acad. Sci. 171: 5-23). The net potential of this reaction is given by the relationship:

$$\begin{matrix}Δ E = {E}_{HOX} - {E}_{{H}_{2} {O}_{2}} = \frac{RT}{n F} ln \frac{[ {H}_{2} O ] [ {H}^{+} ] [ {X}^{-} ] {[}^{1} {O}_{2}^{*} ]}{[ HOX ] [ {H}_{2} {O}_{2} ]} & \\ & \end{matrix} \qquad ( 10 )$$

The methods of the invention provide a unique two-step phased approach for the decontamination, disinfection, or sterilization of contaminants or microorganisms, such as those found on material surfaces, on human or animal skin and wounds, and in untreated water. In a first phase (Phase 1) of the methods of the invention, a first composition comprising a hypohalite, preferably a hypochlorite such as action sodium hypochlorite, is applied to a target surface, skin, or contaminated water. The alkaline nature of sodium hypochlorite provides an alkaline surfactant-like action. The electrophilic chloronium-like action of hypochlorite oxidizes (dehydrogenates), chlorinates or otherwise destroys chemical toxins or pathogenic biological microorganisms. The concentration of hypohalite employed will depend on the specific requirements for sterilization. However, the potency and duration of activity for this binary system can easily be regulated by the concentration and duration of OCl⁻ exposure (Phase 1 binary action).

In a second phase (Phase 2) of the binary system, a second composition comprising hydrogen peroxide is applied to the hypochlorite-treated target. Peroxide reacts with residual hypochlorite and chloramines products yielding singlet molecular oxygen, a potent broad spectrum electrophilic reactant that can oxygenate a broad spectrum of organic and biological molecules, including chemical toxins and biological organisms. Singlet oxygen is a metastable excited state of oxygen with a potent, but limited reactive lifetime, on the order of several milliseconds. The combustive action of singlet oxygen is limited to a radius of about 0.1 to 0.2 microns from its point of generation. Any unreacted singlet oxygen relaxes to triplet oxygen by emitting a benign infrared photon. Phase 2 action terminates the oxidative chlorinating action of Phase 1, produces a short-lived, but potent, burst of singlet oxygenation, yielding products that include oxidized/oxygenated toxins, dead microbes, and innocuous pH-neutral dilute saline solution.

Hypochlorite, the reactant in the compositions of Phase 1 of the binary system, is a well established decontaminating agent that is highly effective against many of the known chemical and biological warfare agents. Peroxide, the second reactant in the compositions of Phase 2 of the binary system, is also a well known disinfecting agent, although it is not as effective as hypochlorite. In addition to augmenting the potent and well established decontaminating capacity of hypochlorite by adding the potent, short-lived oxygenating (combustive) capacity of singlet oxygen, the binary system allows control over Phase 1 reaction duration. In Phase 2, residual hypochlorite and chloramines are destroyed yielding a dilute saline solution, i.e., a safe decontaminated effluent requiring no or minimal clean up and removal.

As a detoxifying and microbicidal agent, hypochlorite is limited by its controllability, not by its potency. By providing Phase 2 control, the two step methods of the invention allow full realization of the hypochlorite microbicidal capacity. For example, hypochlorite can be employed in this first step at high concentrations for short reaction times, fully realizing its rapid detoxifying and microbicidal potential. Exposing a surface or solution to concentrated hypochlorite insures rapid disinfection and killing that is rapidly terminated in the second step exposure to peroxide (Phase 2).

Contacting the target with hydrogen peroxide (Phase 2) in quantities sufficient to completely react with residual hypochlorite and its chloramines products guarantees termination of Phase 1 reactivity, produces a short-lived burst of singlet oxygenation, and yields an innocuous sterile salt solution. The final concentration of the saline (e.g., sodium chloride or calcium chloride) solution will depend on the concentration of hypohalite (e.g., sodium hypochlorite or calcium hypochlorite) employed for Phase 1 action. While the relatively short-lived burst of Phase 2 singlet oxygenation provides additional microbicidal reactivity compared to hypohalite (hypochlorite) treatments alone, the ability of the peroxide treatment to temper or terminate the reactive exposure of the hypohalite treatment provides a significant benefit in the practice of the invention. The two-step methods of the invention thus extend and complement hypochlorite germicidal action by: (1) introducing a short-lived singlet oxidation Phase 2 to the hypochlorite germicidal action, (2) providing Phase 2 control over of Phase 1 activity (reaction duration), and (3) yielding innocuous saline solution as product. A key advantage of this method over conventional hypochlorite treatment is that it provides temporal control over hypochlorite reaction time and yields innocuous saline solution as a reaction product.

The two reactive agents of the binary system, when appropriately combined, provide a rapid, effective means of decontamination, disinfection, or sterilization. The methods and compositions of the invention can play a major role in remediation efforts following contamination by a wide variety of chemical and biological agents. The key advantages of this technology over other methods are augmented reactivity against a broad range of targets, control over reaction duration, cost-effectiveness, ready availability, and a safe effluent product stream. The binary system, therefore, provides potent, controlled, broad-spectrum oxidative decontamination of surfaces, skin, or water without residual toxicity.

This technology is ideal for first responders arriving at the scene of a yet uncharacterized chemically or biologically contaminated site. The primary components of the phased binary system are made by established companies with stable product lines, and available from distributors in virtually all geographic locations. The delivery systems needed to apply compositions comprising the primary components are also available as standard off-the-shelf items.

Definitions

The following is a list of terms and their definitions as used in the specification and the claims:

The term “antisepsis” is defined as substantial reduction of microbial content.

The term “anaerobic” or “substantially anaerobic” means in the absence of oxygen or substantially in the absence of oxygen.

The term “decontamination” means antisepsis, disinfection, or sterilization of microorganisms and the detoxification of susceptible chemical agents.

The term “disinfection” implies destruction of all viable microorganisms, except for spores, particularly microorganisms capable of causing disease.

The term “sterilization” means the complete elimination of all viable microorganisms, including spores.

The term “surface” is the defined the outermost boundary of an inanimate object and/or animate object and subject.

Abbreviations and their corresponding meanings include:

CFU=colony forming units

g=gram(s)

mg=milligram(s)

ml or mL=milliliter(s)

mm=millimeter(s)

mM=millimolar

MPO=myeloperoxidase

nmol=nanomole(s)

pmol=picomole(s)

ppm=parts per million

RT=room temperature

U=unit(s)

μg=microgram(s)

μL or μl=microliter(s)

μM=micromolar

### BRIEF DESCRIPTION OF THE DRAWINGS

The foregoing aspects and many of the advantages of this invention will become more readily appreciated and better understood by reference to the following detailed description, when taken in conjunction with the accompanying drawings, wherein:

FIG. 1 illustrates the UV spectra of a solution of sodium hypochlorite at 2 mM concentration.

FIG. 2 illustrates the UV spectra of a 10 mM solution of hydrogen peroxide.

FIG. 3 illustrates the UV spectra of a 2 mM solution of hydrogen peroxide.

FIG. 4 illustrates the UV Spectra of an equimolar solution of sodium hypochlorite and hydrogen peroxide at 2 mM.

FIG. 5 illustrates the UV spectra of a solution containing 2 mM sodium hypochlorite and 10 mM of hydrogen peroxide; a 5-fold molar excess of hydrogen peroxide.

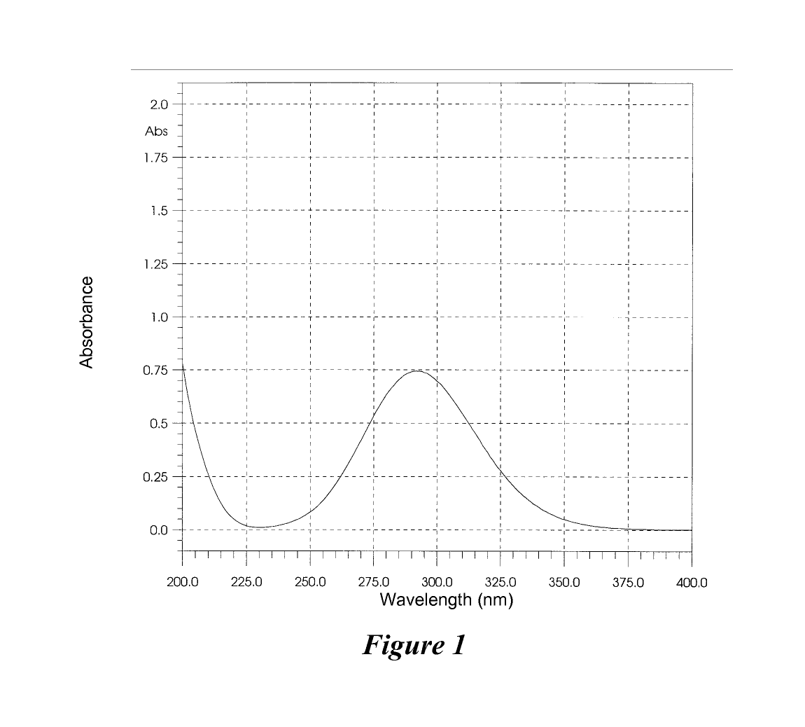

Please see Example 9 for more detailed descriptions of the experiments leading to the UV spectra illustrated in FIGS. 1-5.

### DETAILED DESCRIPTION OF THE INVENTION

The present invention relates to binary methods and compositions comprising hypohalite (preferably a hypochlorite, such as sodium hypochlorite) and peroxide (preferably hydrogen peroxide) directed to the killing of parasites, bacteria, fungi, yeasts, and prions, the oxidation of toxins, and the preparation of potable water. The binary methods and compositions extend the microbicidal potency of conventional hypochlorite by providing additional singlet molecular oxygen generated in situ, and offer more control over reactive chlorination exposure than hypochlorite alone. This combination provides a highly effective disinfecting and decontaminating agent, capable of disinfection, sterilization, detoxification, and/or deactivation of most sources of biological contamination and many chemical toxins.

In the binary system of the invention a hypohalite solution, such as a sodium hypochlorite solution, is applied to a target, such as contaminated surface, skin, or water to be treated, at a sufficient concentration to rapidly oxidize (dehydrogenate) and chlorinate toxins or microbial targets. Subsequently, the surface, skin, or water is treated by addition an aqueous solution of peroxide, such as hydrogen peroxide, at a concentration sufficient to react with residual hypochlorite and chlorinated intermediate products to produce singlet oxygen (¹O₂*), a highly potent electrophilic reactant.

In some aspects, the present invention provides methods of decontaminating a surface or liquid target comprising contacting the target with a first composition comprising hypohalite for a first treatment time, and then contacting the target with a second composition comprising a sufficient amount of peroxide to react with substantially all of the hypohalite in the first composition for a second treatment time.

In order to ensure that substantially all of the hypohalite of the first composition is reacted and effectively neutralized, the molar ratio of hypohalite in the first composition to peroxide in the second composition should generally be 1:1 or less, in some cases 1:2 or less, and in other cases 1:4 or less.

Hypohalites useful in the first composition of the invention include alkali metal and alkaline earth salts of hypohalite, and species capable of producing the desired hypohalite in situ. Preferably the hypohalite is hypochlorite. Examples of suitable hypochlorites may include alkali metal hypochlorites such as sodium hypochlorite, calcium hypochlorite, lithium hypochlorite, and the like, with sodium hypochlorite being preferred. In some embodiments, the first hypohalite composition comprises an aqueous solution of hypohalite. The concentration of hypochlorite in the first composition will vary depending on the nature of the contamination and the target to be treated. In representative embodiments, the first hypohalite composition will comprise hypohalite at a concentration from 0.0001 mM to 5 M, in some cases the concentration is from 0.001 mM to about 1 M, and in other cases the concentration is from 0.01 mM to about 700 mM.

Examples of suitable peroxides useful in the second composition of the invention include hydrogen peroxide, metal peroxides, as well as alkali and alkaline earth metal peroxides, and agents capable of generating peroxide in situ. Specific non-limiting examples include hydrogen peroxide and alkyl hydroperoxides of the formula (R—OOH) wherein R is a hydrogen or a short chain alkyl group having from 1 to 3 carbon atoms, which include barium peroxide, lithium peroxide, magnesium peroxide, nickel peroxide, zinc peroxide, potassium peroxide, sodium peroxide, and the like, with hydrogen peroxide and sodium peroxide being preferred, and hydrogen peroxide being particularly preferred. In some embodiments, the second peroxide composition comprises an aqueous solution of peroxide, preferably an alkali metal peroxide, such as sodium peroxide. In other embodiments, the peroxide in the second composition is hydrogen peroxide. The concentration of the peroxide will vary depending on the reaction conditions and the amount of hypohalite employed in the first composition. In representative embodiments the concentration of hydrogen peroxide in the second composition is from 0.001 mM to 10 M, in some cases from 0.01 mM to 1 M, and in other cases from 0.1 mM to 880 mM.

Aqueous solutions of sodium hypochlorite and hydrogen peroxide react in a diffusion controlled process to produce oxygen. The individual chemical reactions are as follows:
NaOCl+H₂O→NaOH+HOCl
HOCl+H₂O₂→H₂O+¹O₂*+HCl
NaOH+HCl→NaCl+H₂O
¹O₂*→³O₂↑+photon (hν)

The net reaction, i.e., the sum of the above, is shown below.
NaOCl+H₂O₂→NaCl+H₂O+³O₂↑+photon (hν)

The concentration of hypochlorite solution in Step 1 of the binary methods of the invention may vary from 0.0001 mM to 5 M and the concentration of hydrogen peroxide from 0.001 mM to 10 M. Depending on the concentration of hypochlorite the treatment time will vary; the higher the concentration the shorter the time for equivalent outcome. Depending on the type of surface and the level of contamination the treatment time with first composition of the invention could be as short as 1 minute. Ideally the concentration of hydrogen peroxide should be at least equimolar to that of hypochlorite. No limit on the time of treatment is relevant because it is involved in the neutralization of hypochlorite.

In an embodiment, the hypohalite in the first composition is sodium hypochlorite and the peroxide in the second composition is hydrogen peroxide. In some embodiments the concentration of sodium hypochlorite is from about 0.0001 mM to about 5 M and the concentration of hydrogen peroxide is from about 0.001 mM to about 10 M. In other embodiments, the concentration of sodium hypochlorite is from about 0.001 mM to about 1 M and the concentration of hydrogen peroxide is from about 0.01 mM to about 1 M.

The optimum duration for the hypohalite composition to remain in contact with the target (i.e., the first treatment time) prior to contact of the target with the second peroxide composition may vary widely depending on the nature of the target to be treated, the source of contamination, and the amount of hypohalite used in Phase 1 of the treatment. In some embodiments, the first treatment time will be at least 1 minute, and in others at least 5 minutes. In yet other embodiments the first treatment time will be at least 10 minutes. Optimally, the second (peroxide) composition will remain in contact with the target for sufficient period of time (the second treatment time) for the peroxide composition to react with substantially all the hypohalite and chloramines products in the first composition. Since the result of the reaction is neutralization of the hypochlorite and the formation of saline, the second treatment time will be at least 5 seconds, in other cases at least 30 seconds, and in yet other cases at least 30 minutes, and may last indefinitely.

In some embodiments, the methods of the invention are used for decontaminating a target contaminated with a pathogenic microorganism, such as a bacterium, fungi, yeast, virus, or prion. The method can also be used to decontaminate a surface or liquid target contaminated with a pathogenic microorganism in vegetative or spore form. The microorganism may be in spore form, preferably from the group consisting of Bacillus, Clostridia, and Sporosarcina, and more preferably from the group consisting of Bacillus anthracis, Bacillus subtilis, Bacillus thuringiensis, and Clostridia botulinum.

The methods can be used to decontaminate several types of targets. In one embodiment, the target is an animal, preferably human, and the surface target is skin or hair. The method can also be used to decontaminate a variety of inanimate objects, such as vertical and horizontal surfaces on buildings and equipment. The target may also be liquid, such as contaminated water.

In other aspects, the solutions of Phase 1 or Phase 2 of the binary system of the invention may comprise additional additives to modify solution properties, as may be desired as to increase the thoroughness and duration of contact between the individual solutions of the binary system and the target object surfaces, or for other purposes.

By including detergents, surfactants, or other agents in the solutions, the oxygen that is produced by the method of the invention is entrained to produce foam. The characteristics of this foam can be controlled by appropriate choice of additives. The physical structure of the foam retards the drain time such that effective contact duration on non-vertical surfaces is increased. The additives may facilitate the treatment of most surfaces (solid, semi-porous, irregular) and target objects being decontaminated by increasing the thoroughness and duration of contact between the primary compositions of the invention and the target object surfaces. This increased or improved contact can be achieved in several ways:

Longer reaction times increase the transfer of nascent chlorine to the target, thus requiring that the binary system components remain in contact with the target as long as possible once they are applied. In the case of vertical surfaces, simple run-off drastically limits the duration of contact therefore, additives can be used to address this issue. The drain-time from vertical surfaces can be increased by augmenting solution viscosity. Gelling agents, thixotropic agents, and viscosity enhancing agents can be used to prolong the contact time of hypochlorite and hydrogen peroxide with the surface. A few examples of these types of agents include but are not limited to, amorphous colloidal silica gel, polyethylene glycols, methoxypolyethylene glycols, ethylcellulose, methylcellulose, hydroxypropylcellulose, hydroxypropylmethylcellulose, and hydroxyethylcellulose, gelatin, and alginates.

The first composition, the second composition, or both the first and second compositions, may further comprise one or more surfactants, detergents, or co-solvents. Useful surfactants and detergent include non-ionic, anionic, cationic zwitter-ionic surfactants, and detergents. Representative examples of surfactants include polyoxyethylene sorbitan esters, polyoxyethylene ethers, alkyl polyglucosides, alcohol or phenol ethoxylates, alkylamine ethoxylates, alkylarylether sulfates or sulfonates, alkyldiphenyloxide disulfonates, and alkylarylammonium halides. More preferably the surfactant is selected from the group consisting of polyoxyethylene sorbitan monooleate, polyethoxy cetylether, sodium octylphenoxypolyethoxyethyl sulfonate, sodium dodecyl sulfate, sodium deoxycholate, benzalkonium chloride, dodecyltrimethylammonium bromide, polyoxyl castor oil, polyoxyl hydrogenated castor oil, polyethylene-polypropylene glycol, octyl-beta-D-glucopyranoside, triethyleneglycol monododecylether, and dimethylpalmitylammonio-propane sulfonate.

In other embodiments, the first and/or second compositions may further comprise co-solvents, such as alcohols, glycerols, and glycols. Representative samples include isopropyl alcohol, butanol, glycerin, propylene glycol, and butanediols.

In other embodiments, the first composition, the second composition, or both the first and second compositions, further comprise one or more gelling agents, thixotropic agents, or viscosity enhancing agents, such as amorphous colloidal silica gel, polyethylene glycols, methoxypolyethylene glycols, ethylcellulose, methylcellulose, hydroxypropylcellulose, hydroxypropylmethylcellulose, and hydroxyethylcellulose, gelatin, and alginates.

In other embodiments, the first composition, the second composition, or both the first and second compositions, further comprise one or more detection agents for detecting the coverage of the first and/or second composition of the invention when applied to a target. In some embodiments the first and second detection agents are not the same. In some embodiments, the detection agent may be a colored dye.

In another aspect, the present invention provides for kits for decontaminating a surface or a liquid target comprising a first container containing a first composition comprising hypohalite and a second container containing a second composition comprising peroxide. In some embodiments, the first composition comprises hypochlorite and the second composition that comprises hydrogen peroxide. In other embodiments, the present invention also provides for kits wherein said first composition, said second composition, or both first and second compositions, further comprise one or more surfactants, detergents, co-solvents, gelling agents, thixotropic agents, viscosity enhancing agents, or detection agents. In a preferred embodiment, the first composition comprises a first detection agent and the second composition comprises a second detection agent wherein the first and second detection agents are not the same.

In other aspects, the compositions of the invention may further comprise agents that provide a signal indicating that solutions comprising the primary components were properly applied, in the right order, in the right amount, and when applied to surfaces, are spread evenly, to ensure that the primary components can react with the target microorganisms or compounds. The agents themselves can be inert, such as colloidal paint suspensions, that readily indicate the area and intensity of a solution comprising a primary component sprayed on a large surface. The solution comprising the first primary component, for example, may further comprise a first colored agent, and the solution comprising the second primary component may further comprise a second colored agent. On a sprayed surface, the two colored agents mix to produce a visual effect providing assurance that the two solutions were evenly applied in the proper order. For example, a surface treated with a Phase 1 hypohalite composition comprising a blue agent, and then treated with a Phase 2 peroxide composition comprising a yellow agent, for example, might provide the visual effect of a surface being green, where the surface has been evenly treated with both compositions. Gaps in coverage areas can quickly be noted, and the surfaces treated again, if necessary, to ensure the decontamination procedure is complete.

Other more sophisticated detection agents may be used, including those that provide readouts reflecting the concentration of either of the primary components, or are independent of the concentration of such components, depending on its intended application. Some agents, for example, may be sensitive to pH, such as dye indicators, which when the reaction between the primary components is complete, change colors or become colorless. Other agents are contemplated, which may change colors or become colorless upon exposure to air. A newly-treated wet surface, for example, may be one color, but turn colorless upon drying. Other agents are also contemplated that may be detectable with systems, such as handheld UV lamps, and the like, that permit the viewing of a detection agent on treated surface that is not ordinarily visible to unaided human eye. Various other supplementary agents that facilitate the detection of solutions comprising the primary components, and optionally provide a readout of the primary chemical reactions that result in decontamination of a solution or a surface, are intended to be within the spirit and scope of the invention.

The first and second compositions of the invention may be applied by an of a number of techniques using common equipment. These include immersion of the objects, if possible, in separate tanks containing the first composition, and then the second composition. Other methods include spraying or painting the target surfaces using equipment well known to those skilled in the art. The surfaces may be treated several times, if necessary, to complete the procedure for difficult to treat surfaces, such as irregular or porous surfaces or those containing thick biofilms. Ordinarily, multiple treatments would not be required or expected.

The potency and duration of activity of this two-step methods of the invention can be regulated by the concentration and duration of hypochlorite exposure (Phase 1 action). Depending on the contaminating agent and operational conditions, and after the desired optimal exposure time, the chlorinating and dehydrogenating activities are terminated by the addition of hydrogen peroxide (Phase 2 action) initiating a burst of singlet oxygenating activity. In the process of generating singlet oxygen, residual hypochlorite and chloramines are destroyed to produce a dilute innocuous saline solution.

Singlet oxygen, with a higher oxidation potential than either hypochlorite or hydrogen peroxide alone, is a potent electrophilic oxygenating agent capable of reacting with a broad spectrum of electron-rich compounds. These include olefins, dienes, sulphides, aromatics, hetero-aromatics, terpenes, steroids, fatty acids, flavones, amino acids, proteins, nucleic acids, blood, bile pigments, and synthetic polymers. Most of the reactions proceed by way of 4+2 and 2+2 cycloadditions, ene reactions, and oxygenation of electron rich heteroatoms such as nitrogen, sulfur, and phosphorus.

Unlike hypochlorite and chloramines, the singlet oxygen is in a metastable, electronically excited state with a finite reactive lifetime. Singlet oxygen has a reported half-life in aqueous solution of 1 to 3 microseconds and a radius of reactivity of about 0.2 micron or less. If it does not react with target molecules or microorganisms near its point of generation, it relaxes to the triplet ground state by emitting an infrared photon. In the case of the two-stage binary system, there is an effective boost to the efficiency of use of singlet oxygen given its formation from the chloramines on the surface of the target; this proximity markedly increases the likelihood of singlet oxygen reacting with that target. The short-lived burst of singlet oxygen, provides a mechanism for direct oxygenation of the target toxin or microbe. As such, Phase 2 peroxide application effectively terminates the hypochlorite action of Phase 1, generates a burst of direct singlet oxygenation, and yields the dilute non-toxic saline as a final product.

The mechanism of action of the binary system, for targets containing susceptible nitrogen groups, proceeds via the nascent chlorine transfer to generate N-chloro compounds and subsequent transfer of the oxidation potential of these N-chloro compounds to the peroxide yielding singlet oxygen. The microbicidal activity of singlet oxygen is well established, and is most likely related to oxidative destruction of membrane integrity, and/or the oxidative inhibition of the enzymes required for metabolic function.

The initial hypochlorite step of the binary system is itself capable of deactivating most of the known chemical weapon agents. For example, the phosphonyl fluoride, GB, is directly hydrolyzed by the alkaline hypochlorite solution. The phosphonyl thiols such as VX undergo a two-stage deactivation process whereby the sulfur is oxidized by the sodium hypochlorite and the resultant phosphonylsulfoxide undergoes rapid alkaline hydrolysis to yield non-toxic products. Mustard agents such as HD are similarly oxidized and the products formed are subsequently subjected to further reaction by singlet oxygen to yield low molecular weight sulfonic acids and inorganic sulfate salts.

Under properly controlled conditions of concentration and reaction time, the binary system can be directly applied for rapid skin disinfection or sterilization. At higher concentrations and with increased exposure duration, this binary formulation system can be applied to sterilization of inert surfaces and destruction of biofilms. For particularly stubborn biofilms, the application of hypohalite compositions of the invention followed by peroxide compositions of the invention can be repeated.

The binary formulations can also be modified, for example, by adjustment of pH, and judicious selection of the counterion for hypochlorite, i.e., sodium hypochlorite, to yield sodium chloride.

The water to be treated should first be coarsely filtered to remove particulate matter and decrease the biomass, if such removal is necessary. In an illustrative embodiment of this aspect of the invention, NaOCl (or Ca(OCl)₂) is added to water to be treated to yield a final concentration of about 0.5% (w/v), the solution is well mixed and allowed to react for at least one hour. An aqueous solution of hydrogen peroxide is then added in an amount equivalent to the concentration of hypochlorite (about 0.5% (w/v), and the solution well mixed. Residual peroxide can be removed by addition of catalase. This treatment will produce drinkable water with a salinity of about half normal saline (0.5%). If the water to be treated is relatively clean (has low biomass), proportionally less NaOCl and hydrogen peroxide can be employed.

It is contemplated that the two-step binary system can be advantageously utilized in a wide range of applications, where decontamination, disinfection, or sterilization is desired, as provided by the representative examples below.

Human Skin Decontamination

Povidone-iodine is currently one of the most effective antiseptic agents uses to facilitate the rapid decontamination of skin (Mimoz et al., 1999, Ann Intern Med. 131(11): 834-7). Two percent chlorhexidine gluconate has been shown to be superior in preventing catheter-related infections compared to 70% isopropyl alcohol and 10% povidone-iodine (Maki, Ringer, Alvarado, 1991, Lancet, 338(8763): 339-43). None of these agents, however, are sufficiently potent or reliable to produce reliable skin disinfection or sterilization, particularly when the skin is contaminated with unknown, and even many known, microorganisms. As such, contamination of blood cultures continues to be a costly problem in medical care.

The keratinized epithelium of intact skin provides usually provides adequate protection against short exposure to relatively high concentrations of sodium hypochlorite solutions. Sensitive and compromised skin (eyes, mucous membranes, wounds), of course, are exceptions. However, even low concentrations of hypochlorite provide much greater antiseptic action than povidone-iodine or chlorhexidine.

The methods and compositions of the invention can be used to rapidly decontaminate the skin of persons requiring immediate medical attention after exposure to chemical or biological agents. The methods and compositions are also ideal for decontaminating skin surfaces in various field and office procedures, such as intravenous line insertion and attachment of monitoring devices.

Water Treatment

Current methods of municipal water treatment provide for the adequate treatment for common and well known microorganisms. If a storage reservoir becomes contaminated with bioterrorism agents, however, an effective additional method is needed for water treatment in situ. Treatment of water supplies contaminated with unknown agents by bleach alone may be insufficient and present new problems relating to toxicity from the action of residual hypochlorite. The methods and compositions of the present invention, however, can facilitate this function at the point of distribution, since Phase 1 treatment with hypochlorite followed by Phase 2 peroxide, which provides an additional burst of oxygenation activity, effectively deactivates both chemical toxins and biological agents.

Depending on the type of water treatment process used, the decontamination can be achieved by first adding hypochlorite and then adding peroxide to clearwells, storage holding tanks, small reservoirs, flash mix basins, and the like. At points of distribution, sections of the water main may be isolated and the contents treated in a batch-wise fashion. The decontamination methods can also performed at the point of use.

The water to be treated should first be coarsely filtered to remove particulate matter and decrease the biomass, if such removal is necessary. In an illustrative embodiment of this aspect of the invention, NaOCl (or Ca(OCl)₂) is added to water to be treated to yield a final concentration of about 0.5% (w/v), the solution is well mixed and allowed to react for at least one hour. An aqueous solution of hydrogen peroxide is then added in an amount equivalent to the concentration of hypochlorite (about 0.5% (w/v), and the solution well mixed. Residual peroxide can be removed by addition of catalase. This treatment will produce drinkable water with a salinity of about half normal saline (0.5%). If the water to be treated is relatively clean (has low biomass), proportionally less NaOCl and hydrogen peroxide can be employed.

Decontamination of Surfaces

The methods and compositions of the two step binary system of the invention can be used to facilitate the decontamination, disinfection, or sterilization of all types of material surfaces. The two step binary system of the invention may be applied to relatively small surfaces such as clothing, laboratory equipment, or medical equipment or devices, or to relatively large surfaces, such as equipment, buildings, or land, including tarmacs, docks, and vehicles, producing environmentally-compatible waste products.

The methods and compositions of the invention can also be modified to use compositions comprising the primary components, hypochlorite and peroxide, which facilitate their application to vertical, porous, and non-porous surfaces. Gels, gums, foams, and other agents which modify the viscosity of a solution, or facilitate the penetration of a solution into absorbent materials are contemplated. The modified compositions provide greater penetrability and longer contact time than unmodified aqueous solutions comprising hypochlorite or peroxide alone. The resulting methods and compositions provide economic benefits in terms of availability and cost of supplies, labor cost, and management of downstream waste products.

### EXAMPLES

The foregoing discussion may be better understood in connection with the following representative examples which will illustrate the additional microbicidal action associated with Phase 2 peroxide exposure. These examples are designed to illustrate Phase 2 singlet oxygenation. Although it will be understood that the invention is not limited to these specific examples, and in most applications the Phase 1 hypochlorite exposure will be sufficient to produce rapid and complete detoxifying or microbicidal action but insufficient to damage the skin or surface treated. Likewise, the concentration of peroxide use for Phase 2 treatment will be higher in proportion to the higher hypochlorite concentrations used for Phase 1 treatment. Various other examples will be apparent to the person skilled in the art after reading the present disclosure without departing from the spirit and scope of the invention. It is intended that all such other examples be included within the scope of the appended claims.

Unless noted otherwise, all other specialty chemicals were obtained from Sigma (St. Louis, Mo.). All parts are by weight, and temperatures are in degrees centigrade (° C.), unless otherwise indicated.

### Example 1

### Microbicidal Activity Against Staphylococcus Aureus

The augmented microbicidal activity of the binary system against Staphylococcus aureus compared to that of sodium hypochlorite solution alone or hydrogen peroxide solution alone was demonstrated as follows.

Materials

Bacterial suspensions, specifically Staphylococcus aureus (ATCC 6538) in this example, were prepared by the shake flask method to achieve late log to early stationary phase growth. Bacteria were grown 24 hours in trypticase soy broth (TSB) at 35° C. The cultures were centrifuged at 4,000 rpm for 10 minutes and the supernatants removed. The pellet was collected and washed twice with sterile 0.9% normal saline. The washed microorganisms were suspended and diluted with normal saline to a 3 McFarland standard, i.e., approximately 10⁹ bacteria colony forming units (CFU) per ml. Actual colony counts are confirmed by serial dilutions (10⁻¹ to 10⁻⁵ or 10⁻⁶) plated on trypticase soy agar (TSA) and incubated overnight at 35° C. One hundred microliters of organisms from a stock of 10⁷ CFU/ml were used to obtain an approximate final working target inoculum of 10⁶ CFU. The microbicidal test is conducted in a vial containing 1.4 ml of final reaction mixture after neutralization.

Liquid Bleach (sodium hypochlorite 5.25%) (Fisher, Cat # S66362) was diluted in sterile water to prepare the sodium hypochlorite solutions at desired concentrations.

Hydrogen peroxide 30% (Fisher, Cat # H325-500) was diluted in sterile water to prepare the hydrogen peroxide concentrations required for this study.

Sodium Thiosulfate (Fisher, Cat # S-556) was used as a 2.4% solution.

Catalase (Sigma, Cat # C-40) was prepared as a 1% stock solution in sterile 0.9% normal saline.

Methods

Using sterile techniques, hypochlorite and hydrogen peroxide solutions were prepared at concentrations indicated in Table 1. Each solution was used alone for the individual controls. Organism suspensions were used to achieve a final target concentration of 2-3×10⁶ CFU per ml. For the binary system treatments, sodium hypochlorite solution and hydrogen peroxide solution, non-acidified or acidified, were sequentially added to the microorganisms. Acidified hydrogen peroxide solution was obtained by the addition of 1.0% v/v of 0.001 N hydrochloride solution. After each addition, the resulting mixture was allowed to remain in contact with the organisms for a set amount of time at room temperature (about 22° C.), as listed in the Tables 1 and 2. At the end of the treatment time, the mixtures were neutralized with 200 to 500 microliters of thiosulfate solution (2.4%) to quench the activity of sodium hypochlorite and then treated with 100 to 200 microliters of a 1% catalase solution, containing a minimum of 100 units/ul, to quench the hydrogen peroxide activity. If needed, an appropriate volume of sterile saline was added to bring the final volume of reaction mixture to 1.4 ml after neutralization. Serial dilution plate counts were performed from the contents of each vial in sterile saline and inoculated onto TSA for quantitative culture. Plates were then incubated at 37° C. and counts taken at 24 hours. After incubation, the surviving colony forming units (CFU) were counted as a measure of the viability of the organisms and results compared to an inoculum control. The results are shown in Table 1 and Table 2, below.

Results

Table 1 presents the results of hypochlorite solution or hydrogen peroxide solution alone against the Gram-positive bacteria Staphylococcus aureus. These data quantify the activity of hypochlorite solution or hydrogen peroxide solution alone and thus serve as reference data for comparing the microbicidal activity of the binary system, which is presented in Table 2.

TABLE 1
Microbicidal Activity of Sodium Hypochlorite Solution
or Hydrogen Peroxide Solution Alone Against Staphylococcus aureus

| NaOCl | NaOCl | H₂O₂ | H₂O₂ | Starting | | Log 10 | |
|---|---|---|---|---|---|---|---|
| Conc | time | Conc | Time | Inoculum | Total Viability | (CFU + 1) | |
| mM | min | mM | (min) | (CFU) | (CFU) | Survivors | Log Reduction |
| 705.27 | 5 | | | 1600000 | 0 | 0.0 | 6.2 |
| 70.53 | 5 | | | 1600000 | 0 | 0.0 | 6.2 |
| 7.05 | 5 | | | 1600000 | 0 | 0.0 | 6.2 |
| 0.71 | 5 | | | 1600000 | 0 | 0.0 | 6.2 |
| 0.07 | 5 | | | 1600000 | 4 | 0.7 | 5.5 |
| 705.27 | 15 | | | 1600000 | 0 | 0.0 | 6.2 |
| 70.53 | 15 | | | 1600000 | 0 | 0.0 | 6.2 |
| 7.05 | 15 | | | 1600000 | 0 | 0.0 | 6.2 |
| 0.71 | 15 | | | 1600000 | 0 | 0.0 | 6.2 |
| 0.07 | 15 | | | 1600000 | 7 | 0.9 | 5.3 |
| 705.27 | 30 | | | 1600000 | 0 | 0.0 | 6.2 |
| 70.53 | 30 | | | 1600000 | 0 | 0.0 | 6.2 |
| 7.05 | 30 | | | 1600000 | 0 | 0.0 | 6.2 |
| 0.71 | 30 | | | 1600000 | 0 | 0.0 | 6.2 |
| 0.07 | 30 | | | 1600000 | 0 | 0.0 | 6.2 |
| | | 8821 | 5 | 1600000 | 0 | 0.0 | 6.2 |
| | | 2940 | 5 | 1600000 | 0 | 0.0 | 6.2 |
| | | 882 | 5 | 1600000 | 12040 | 4.1 | 2.1 |
| | | 294 | 5 | 1600000 | 1288000 | 6.1 | 0.1 |
| | | 88 | 5 | 1600000 | 1694000 | 6.2 | 0.0 |
| | | 8821 | 15 | 1600000 | 0 | 0.0 | 6.2 |
| | | 2940 | 15 | 1600000 | 0 | 0.0 | 6.2 |
| | | 882 | 15 | 1600000 | 0 | 0.0 | 6.2 |
| | | 294 | 15 | 1600000 | 1190000 | 6.1 | 0.1 |
| | | 88 | 15 | 1600000 | 2044000 | 6.3 | −0.1 |
| | | 8821 | 30 | 1600000 | 0 | 0.0 | 6.2 |
| | | 2940 | 30 | 1600000 | 0 | 0.0 | 6.2 |
| | | 882 | 30 | 1600000 | 0 | 0.0 | 6.2 |
| | | 294 | 30 | 1600000 | 21 | 1.3 | 4.9 |
| | | 88 | 30 | 1600000 | 1792000 | 6.3 | −0.1 |

Note:
The starting inoculum was 6.2 log₁₀ CFU. The volumes of sodium hypochlorite, peroxide, and saline used were 0.5 ml each.

As shown in Table 1, the microbicidal activity of both sodium hypochlorite and hydrogen peroxide are time and concentration dependent; increasing contact time as well as increasing concentration enhances microbicidal activity. Within 5 minutes, 8821 and 2940 mM hydrogen peroxide provide complete kill within 5 minutes of a 6.2 log₁₀ CFU inoculum; intermediate concentrations result in partial kill and 88 mM hydrogen peroxide exhibits no microbicidal activity within 30 minutes. Therefore, peroxide concentrations equal to or lower than 88 mM were used for the binary system evaluation in order to eliminate the possibility that any microbicidal activity could primarily be attributable to hydrogen peroxide.

Table 1 also shows that the lowest concentration of sodium hypochlorite tested, 0.07 mM, resulted in only four or seven CFU survivors from the starting inoculum of 6.2 log₁₀ CFU after respective five or 15 minutes of organism treatment time. Therefore, starting at 0.07 mM, lower concentrations of hypochlorite were used to demonstrate the augmented microbicidal activity of the binary system when compared with the same 15 minutes of exposure to sodium hypochlorite alone. After an initial 15 minutes exposure of the organisms to sodium hypochlorite, either hydrogen peroxide or acidified hydrogen peroxide was added. After an additional 30 minutes, the binary system mixture was then neutralized using 2.4% thiosulfate followed by 1% catalase as described in the Methods Section. It should be noted that the hydrogen peroxide concentrations used for this demonstration of the binary system were significantly lower than the lowest peroxide concentration tested as shown in Table 1.

Table 2 below presents the results obtained after execution of the binary system protocol along with appropriate hypochlorite alone or peroxide alone treatments as controls.

TABLE 2
Microbicidal Activity of Binary System Treatments
Against Staphylococcus aureus

| NaOCl | NaOCl | H₂O₂ | H₂O₂ | H₂O₂/H+ | H₂O₂ | Saline | Starting | Total | Log 10 | |
|---|---|---|---|---|---|---|---|---|---|---|
| Conc | time | Conc | Time | Conc | Time | Time | Inoculum | Viability | (CFU + 1) | Log |
| mM | min | mM | (min) | mM | (min) | (min) | (CFU) | (CFU) | Survivors | Reduction |
| 0.07 | 15 | 0.35 | 30 | | | | 2530000 | 0 | 0.0 | 6.4 |
| 0.06 | 15 | 0.3 | 30 | | | | 2530000 | 0 | 0.0 | 6.4 |
| 0.05 | 15 | 0.25 | 30 | | | | 2530000 | 1 | 0.4 | 6.0 |
| 0.04 | 15 | 0.2 | 30 | | | | 2530000 | 21 | 1.3 | 5.1 |
| 0.03 | 15 | 0.15 | 30 | | | | 2530000 | 78 | 1.9 | 4.5 |
| 0.02 | 15 | 0.1 | 30 | | | | 2530000 | 127 | 2.1 | 4.3 |
| 0.07 | 15 | | | 0.35 | 30 | | 2530000 | 0 | 0.0 | 6.4 |
| 0.06 | 15 | | | 0.3 | 30 | | 2530000 | 0 | 0.0 | 6.4 |
| 0.05 | 15 | | | 0.25 | 30 | | 2530000 | 1 | 0.4 | 6.0 |
| 0.04 | 15 | | | 0.2 | 30 | | 2530000 | 1 | 0.4 | 6.0 |
| 0.03 | 15 | | | 0.15 | 30 | | 2530000 | 8 | 1.0 | 5.4 |
| 0.02 | 15 | | | 0.1 | 30 | | 2530000 | 132 | 2.1 | 4.3 |
| 0.07 | 15 | | | | | | 2530000 | 0 | 0.0 | 6.4 |
| 0.06 | 15 | | | | | | 2530000 | 0 | 0.0 | 6.4 |
| 0.05 | 15 | | | | | | 2530000 | 17 | 1.3 | 5.1 |
| 0.04 | 15 | | | | | | 2530000 | 167 | 2.2 | 4.2 |
| 0.03 | 15 | | | | | | 2530000 | 784 | 2.9 | 3.5 |
| 0.02 | 15 | | | | | | 2530000 | 1288 | 3.1 | 3.3 |
| 0.07 | 15 | | | | | 30 | 2530000 | 0 | 0.0 | 6.4 |
| 0.06 | 15 | | | | | 30 | 2530000 | 7 | 0.9 | 5.5 |
| 0.05 | 15 | | | | | 30 | 2530000 | 13 | 1.1 | 5.3 |
| 0.04 | 15 | | | | | 30 | 2530000 | 18 | 1.3 | 5.1 |
| 0.03 | 15 | | | | | 30 | 2530000 | 260 | 2.4 | 4.0 |
| 0.02 | 15 | | | | | 30 | 2530000 | 1092 | 3.0 | 3.4 |
| | | | | 0.35 | 30 | | 2530000 | 1708000 | 6.2 | 0.2 |
| | | 0.35 | 30 | | | | 2530000 | 1764000 | 6.2 | 0.2 |
| 0.05 | 0 | 88.2 | 30 | | | | 1390000 | 1918000 | 6.3 | −0.1 |

Note:
The starting inoculum was 6.4 log₁₀. The volumes of sodium hypochlorite, peroxide, and saline were 0.5 ml each. H₂O₂/H⁺ corresponds to binary system treatment using acidified peroxide and H₂O₂ corresponds to binary system treatment using peroxide. The starting inoculum for the treatment shown in the last line was 6.4 log₁₀.

The binary system where the microorganisms are treated with sodium hypochlorite for a first treatment period, followed by a treatment with hydrogen peroxide in a second treatment period, demonstrates enhanced microbicidal activity compared to that from 15 minutes of exposure to sodium hypochlorite alone. Similar enhancements are seen compared with controls held for 45 minutes with an intermediate dilution step. Specifically, hypochlorite followed by peroxide in the binary system compared to hypochlorite followed by saline also demonstrated an 8-fold reduction in survivors indicating the synergistic nature of the binary system. Moreover, a subsequent treatment with acidified hydrogen peroxide demonstrates greater synergistic action when compared with non-acidified peroxide. As expected from the data shown in Table 1, exposure with the highest concentration of hydrogen peroxide used in the binary system tested alone was completely ineffective and produced no kill.

A demonstration of the neutralizing efficiency of hypochlorite by hydrogen peroxide is given in the last line of Table 2. In this experiment, 0.05 mM hypochlorite is neutralized by 88 mM peroxide prior to the addition of the inoculum at 30 minutes post-neutralization, which resulted in no measurable microbicidal activity at 20 minutes. This lack of performance is in contrast with that of the control (0.05 mM of hypochlorite alone, also shown in Table 2) being sufficient to kill all but 17 organisms in 15 minutes.

The results presented in Table 2, can also be viewed as the difference in log reduction between the hypochlorite alone controls and the binary system treatments. These data are collected as such in the summary table below.

TABLE 3
A
Log Reduction Between Hypochlorite Alone
and Binary System Treatments with Non-Acidified Peroxide

| Log Reduction | | | | | | | | |
|---|---|---|---|---|---|---|---|---|
| with | | | | | | | | |
| Non-Acidified | | Non Acidified Peroxide mM | | | | | | |
| Peroxide | | 0.00 | 0.35 | 0.30 | 0.25 | 0.20 | 0.15 | 0.10 |
| NaOCl | 0.07 | 6.40 | 6.40 | | | | | |
| mM | 0.06 | 6.40 | | 6.40 | | | | |
| | 0.05 | 5.15 | | | 6.02 | | | |
| | 0.04 | 4.18 | | | | 5.06 | | |
| | 0.03 | 3.51 | | | | | 4.50 | |
| | 0.02 | 3.29 | | | | | | 4.29 |

B
Log Reduction Between Hypochlorite Alone
and Binary System Treatments with Acidified Peroxide

| Log Reduction | | | | | | | | |
|---|---|---|---|---|---|---|---|---|
| with | | | | | | | | |
| Acidified | | Acidified Peroxide mM | | | | | | |
| Peroxide | | 0.00 | 0.35 | 0.30 | 0.25 | 0.20 | 0.15 | 0.10 |
| NaOCl | 0.07 | 6.40 | 6.40 | | | | | |
| mM | 0.06 | 6.40 | | 6.40 | | | | |
| | 0.05 | 5.15 | | | 6.02 | | | |
| | 0.04 | 4.18 | | | | 6.02 | | |
| | 0.03 | 3.51 | | | | | 5.43 | |
| | 0.02 | 3.29 | | | | | | 4.28 |

C
Differences in Log Reduction Between Hypochlorite Alone
and Binary System Treatments with Non-Acidified Peroxide

| Log Redn | | | | | | | | |
|---|---|---|---|---|---|---|---|---|
| Difference with | | | | | | | | |
| Non-Acidified | | Non Acidified Peroxide mM | | | | | | |
| Peroxide | | 0.00 | 0.35 | 0.30 | 0.25 | 0.20 | 0.15 | 0.10 |
| NaOCl | 0.07 | — | 0.00 | | | | | |
| mM | 0.06 | — | | 0.00 | | | | |
| | 0.05 | — | | | 0.87 | | | |
| | 0.04 | — | | | | 0.88 | | |
| | 0.03 | — | | | | | 1.00 | |
| | 0.02 | — | | | | | | 1.00 |

D
Differences in Log Reduction Between Hypochlorite Alone
and Binary System Treatments with Acidified Peroxide

| Log Redn | | | | | | | | |
|---|---|---|---|---|---|---|---|---|
| Difference with | | | | | | | | |
| Acidified | | Acidified Peroxide mM | | | | | | |
| Peroxide | | 0.00 | 0.35 | 0.30 | 0.25 | 0.20 | 0.15 | 0.10 |
| NaOCl | 0.07 | — | 0.00 | | | | | |
| mM | 0.06 | — | | 0.00 | | | | |
| | 0.05 | — | | | 0.87 | | | |
| | 0.04 | — | | | | 1.84 | | |
| | 0.03 | — | | | | | 1.92 | |
| | 0.02 | — | | | | | | 0.99 |

The data illustrate that where less than complete kill is observed, hypochlorite followed by either added peroxide or added acidified peroxide provides superior kill when compared to that of hypochlorite alone. Specifically, the use of the binary system with non-acidified peroxide gave up to a 1.0 log₁₀ CFU (10-fold) increase in kill, and the use of acidified peroxide gave up to 1.92 log₁₀ CFU (84-fold) increase in kill when compared to equivalent levels of hypochlorite alone.

The binary system microbicidal activity against S. aureus was investigated further using a higher concentration (88 mM) of hydrogen peroxide and a shorter treatment time (15 minutes) than those shown Table 2. These results are presented in Table 4 below.

TABLE 4
Microbicidal Activity of the Binary System
Against Staphylococcus aureus At 88 mM H₂O₂ for 15 Minutes

| NaOCl | NaOCl | H₂O₂ | H₂O₂ | Starting | | Log 10 | |
|---|---|---|---|---|---|---|---|
| Conc | time | Conc | Time | Inoculum | Total Viability | (CFU + 1) | |
| mM | min | mM | (min) | (CFU) | (CFU) | Survivors | Log Reduction |
| 0.04 | 15 | 88 | 15 | 2500000 | 199 | 2.3 | 4.1 |
| 0.03 | 15 | 88 | 15 | 2500000 | 23380 | 4.4 | 2.0 |
| 0.02 | 15 | 88 | 15 | 2500000 | 1694000 | 6.2 | 0.2 |
| 0.04 | 15 | | | 2500000 | 616 | 2.8 | 3.6 |
| 0.03 | 15 | | | 2500000 | 74200 | 4.9 | 1.5 |
| 0.02 | 15 | | | 2500000 | 1890000 | 6.3 | 0.1 |

Note:
The starting inoculum was 6.4 log10. The volumes of sodium hypochlorite, and hydrogen peroxide, were 0.5 ml each. H₂O₂ corresponds to treatment using peroxide.

These data show that 88 mM of peroxide gave 0.5 log reduction improvement over hypochlorite alone after 15 minutes of treatment time for both 0.04 and 0.03 mM hypochlorite. These results demonstrate that this higher concentration of peroxide does not perform as well at 15 minutes as did the lower concentrations of peroxide at 30 minutes as shown in Table 3.

The results presented in Table 4 can also be viewed as the difference in log reduction between the hypochlorite alone controls and the binary system treatments. These data are collected and presented in Table 5 below.

TABLE 5
A
Log Reduction Between Hypochlorite Alone
and Binary System Treatments at 88 mM Hydrogen Peroxide

| | |
|---|---|
| | Non Acidified |
| Log Reduction with Non- | Peroxide mM |

| Acidified Peroxide | | 0.00 | 88.20 |
|---|---|---|---|
| NaOCl mM | 0.04 | 3.61 | 4.10 |
| | 0.03 | 1.53 | 2.03 |
| | 0.02 | 0.12 | 0.17 |

B
Differences in Log Reduction Between Hypochlorite Alone and
Binary System Treatments at 88 mM Hydrogen Peroxide

| | |
|---|---|
| Log Redn Difference | Non Acidified |
| with Non-Acidified | Peroxide mM |

| Peroxide | | 0.00 | 88.20 |
|---|---|---|---|
| NaOCl mM | 0.04 | — | 0.49 |
| | 0.03 | — | 0.50 |
| | 0.02 | — | 0.05 |

This table represents the difference in log reduction between hypochlorite alone and the binary system using 0.04 mM and 0.03 mM hypochlorite. At this shorter time of exposure to hydrogen peroxide, although the concentration of peroxide was increased, the largest difference observed is 0.5 log10. This suggests that the reaction between the chlorinated organisms and the hydrogen peroxide requires more time than the instantaneous reaction between hydrogen peroxide and sodium hypochlorite.

### Example 2

### Microbicidal Activity of the Binary System Against Escherichia coli

The augmented microbicidal activity of the binary system against Escherichia coli (ATCC 25922) when compared to sodium hypochlorite solution alone or hydrogen peroxide alone was demonstrated using the general procedure described in Example 1. In this study, the contact time of the organisms with hydrogen peroxide was reduced from 30 minutes in Example 1 for S. aureus to 5 minutes in the current example against E. coli.

TABLE 6
Microbicidal Activity of the Binary System Against Escherichia coli

| NaOCl | NaOCl | H₂O₂ | H₂O₂ | | | Log 10 | |
|---|---|---|---|---|---|---|---|
| Conc | time | Conc | Time | Starting Inoculum | Total Viability | (CFU + 1) | Log |
| mM | min | mM | (min) | (CFU) | (CFU) | Survivors | Reduction |
| 0.03 | 15 | 88 | 15 | 1630000 | 160 | 2.2 | 4.0 |
| 0.02 | 15 | 88 | 15 | 1630000 | 60200 | 4.8 | 1.4 |
| 0.01 | 15 | 88 | 15 | 1630000 | 103600 | 5.0 | 1.2 |
| 0.03 | 15 | | | 1630000 | 994 | 3.0 | 3.2 |
| 0.02 | 15 | | | 1630000 | 784000 | 5.9 | 0.3 |
| 0.01 | 15 | | | 1630000 | 1218000 | 6.1 | 0.1 |
| | | 88 | 15 | 1630000 | 588000 | 5.8 | 0.4 |

Note:
The starting inoculum was 6.2 log10. The volumes of sodium hypochlorite and hydrogen peroxide, were 0.5 ml each.

As shown in Table 6 above and Table 7 below, the binary system demonstrates enhanced microbicidal activity against E. coli compared to a 15 minute treatment with hypochlorite alone at all three concentrations of hypochlorite tested. As in previous examples, the results presented in Table 6 can also be viewed as the difference in log reduction between the hypochlorite alone controls and the binary system treatments. These data are collected in Table 7, shown below.

TABLE 7
A
Log Reduction Between Hypochlorite Alone
and Binary System Treatments Against Escherichia Coli

| | | Non | |
|---|---|---|---|
| | | Acidified | |
| Log Reduction | | Peroxide | |
| with Non- | | mM | |
| Acidified Peroxide | | 0.00 | 88.20 |
| NaOCl mM | 0.03 | 3.21 | 4.00 |
| | 0.02 | 0.32 | 1.43 |
| | 0.01 | 0.12 | 1.19 |

B
Differences in Log Reduction Between Hypochlorite Alone
and Binary System Treatments Against Escherichia coli

| | | Non | |
|---|---|---|---|
| Log Redn | | Acidified | |
| Difference with | | Peroxide | |
| Non-Acidified | | mM | |
| Peroxide | | 0.00 | 88.20 |
| NaOCl mM | 0.03 | — | 0.79 |
| | 0.02 | — | 1.11 |
| | 0.01 | — | 1.07 |

From this table it is evident that hypochlorite followed by added peroxide gives superior kill when compared to that of hypochlorite alone. Specifically, the use of the binary system gave up to a 1.1 log10 CFU (13-fold) increase in kill when compared to equivalent levels of hypochlorite alone.

### Example 3

### Microbicidal Activity of the Binary System Against Bacillus subtilis Spores

The augmented microbicidal activity of the binary system against Bacillus subtilis when compared to sodium hypochlorite solution alone or hydrogen peroxide solution alone was demonstrated using the general procedure described in Example 1. Suspensions of Bacillus subtilis (ATCC 19659) containing 100% spores, as confirmed by microscopy, were obtained by washing the spun-down cells with 50% ethanol to eliminate the vegetative form. Starting inoculum of approximately 1-3×10⁶ CFU was used as in Example 1.

Table 8 presents the results of hypochlorite solution or hydrogen peroxide solution alone against the spores of the Gram positive bacterium, Bacillus subtilis. These results serve as reference data for comparing the microbicidal activity of the binary system presented in Table 9

TABLE 8
Microbicidal Activity of Sodium Hypochlorite Solution
or Hydrogen Peroxide Alone Against Bacillus Subtilis Spores

| NaOCl | NaOCl | H₂O₂ | H₂O₂ | | Total | Log 10 | |
|---|---|---|---|---|---|---|---|
| Conc | time | Conc | Time | Starting Inoculum | Viability | (CFU + 1) | Log |
| mM | min | mM | (min) | (CFU) | (CFU) | Survivors | Reduction |
| 705.27 | 60 | | | 1865000 | 0 | 0.0 | 6.3 |
| 70.53 | 60 | | | 1865000 | 0 | 0.0 | 6.3 |
| 7.05 | 60 | | | 1865000 | 43 | 1.6 | 4.6 |
| 0.71 | 60 | | | 1865000 | 14840 | 4.2 | 2.1 |
| 0.07 | 60 | | | 1865000 | 2142000 | 6.3 | −0.1 |
| 705.27 | 120 | | | 1865000 | 0 | 0.0 | 6.3 |
| 70.53 | 120 | | | 1865000 | 0 | 0.0 | 6.3 |
| 7.05 | 120 | | | 1865000 | 0 | 0.0 | 6.3 |
| 0.71 | 120 | | | 1865000 | 1526 | 3.2 | 3.1 |
| 0.07 | 120 | | | 1865000 | 1834000 | 6.3 | 0.0 |
| | | 8821 | 60 | 1865000 | 0 | 0.0 | 6.3 |
| | | 2940 | 60 | 1865000 | 588 | 2.8 | 3.5 |
| | | 882 | 60 | 1865000 | 200200 | 5.3 | 1.0 |
| | | 294 | 60 | 1865000 | 1484000 | 6.2 | 0.1 |
| | | 88 | 60 | 1865000 | 1974000 | 6.3 | 0.0 |

Note:
The starting inoculum was 6.3 log₁₀. The volumes of sodium hypochlorite and peroxide, were 0.5 ml each.

As demonstrated in Table 8, only 8821 mM hydrogen peroxide provides complete kill of a 6.3 log10 CFU inoculum of Bacillus subtilis spores within 60 minutes; 2940 and 882 mM hydrogen peroxide concentrations result in partial kill and 294 to 88 mM hydrogen peroxide exhibits no microbicidal activity within 60 minutes. As for Example 1, hydrogen peroxide concentrations below 88 mM were used for the binary system evaluation in order to eliminate the possibility that microbicidal activity could be attributable to hydrogen peroxide.

Concentration of sodium hypochlorite from 70 mM to 0.07 mM yielded partial kill of the starting 6.3 log10 CFU inoculum after 60 minutes of exposure as shown in Table 7. These concentrations and times were used to demonstrate the improved microbicidal activity of the binary system compared to sodium hypochlorite solution alone. After an initial 60 minutes exposure of the organisms to sodium hypochlorite solution, either hydrogen peroxide or acidified hydrogen peroxide was added. The binary system was neutralized 30 minutes after addition of hydrogen peroxide using 2.4% thiosulfate and 1% catalase as described in Example 1. The hydrogen peroxide concentrations used for the binary system were well below the concentration demonstrating microbicidal activity at 30 minutes in Table 8.

Table 9 below presents the results obtained after execution of the binary system protocol along with appropriate hypochlorite alone or peroxide alone treatments as controls on Bacillus subtilis spores.

TABLE 9
Microbicidal Activity of the Binary System Against Bacillus Subtilis Spores

| NaOCl | NaOCl | H₂O₂ | H₂O₂ | H2O2/H+ | H₂O₂ | Starting | Total | Log 10 | |
|---|---|---|---|---|---|---|---|---|---|
| Conc | time | Conc | Time | Conc | Time | Inoculum | Viability | (CFU + 1) | Log |
| mM | (min) | mM | (min) | mM | (min) | (CFU) | (CFU) | Survivors | Reduction |
| 7.05 | 60 | 35.25 | 30 | | | 1120000 | 0 | 0.0 | 6.1 |
| 0.71 | 60 | 3.53 | 30 | | | 1120000 | 3304 | 3.5 | 2.5 |
| 0.07 | 60 | 0.35 | 30 | | | 1120000 | 1554000 | 6.2 | −0.1 |
| 0.71 | 60 | 35.25 | 30 | | | 1120000 | 658 | 2.8 | 3.2 |
| 0.07 | 60 | 35.25 | 30 | | | 1120000 | 868000 | 5.9 | 0.1 |
| 7.05 | 60 | | | 35.25 | 30 | 1120000 | 6 | 0.8 | 5.2 |
| 0.71 | 60 | | | 3.53 | 30 | 1120000 | 2716 | 3.4 | 2.6 |
| 0.07 | 60 | | | 0.35 | 30 | 1120000 | 1442000 | 6.2 | −0.1 |
| 0.71 | 60 | | | 35.25 | 30 | 1120000 | 1078 | 3.0 | 3.0 |
| 0.07 | 60 | | | 35.25 | 30 | 1120000 | 1176000 | 6.1 | 0.0 |
| 7.05 | 60 | | | | | 1120000 | 17 | 1.3 | 4.8 |
| 0.71 | 60 | | | | | 1120000 | 2898 | 3.5 | 2.6 |
| 0.07 | 60 | | | | | 1120000 | 1666000 | 6.2 | −0.2 |
| | | 35.25 | 30 | | | 1120000 | 1358000 | 6.1 | −0.1 |
| | | | | 35.25 | 30 | 1120000 | 1624000 | 6.2 | −0.2 |

Note:
The starting inoculum was 6.1 log₁₀. The volumes of sodium hypochlorite and peroxide were 0.5 ml each. H₂O₂/H⁺ corresponds to binary system treatment using acidified peroxide and H₂O₂ corresponds to treatment using peroxide.

The binary system demonstrates enhanced microbicidal activity compared to that from 60 minutes of exposure to sodium hypochlorite alone. It should be noted that for the same concentration of sodium hypochlorite, an increased concentration of hydrogen peroxide provides a higher level of microbicidal activity although that concentration of peroxide has no microbicidal effect on the spores as demonstrated by the controls. Specifically, 0.7 mM of sodium hypochlorite treated with 35.25 mM of hydrogen peroxide provides 3.2 log10 kill whereas the same 0.7 mM of sodium hypochlorite only provides 2.5 log10 kill when treated with 3.53 mM of hydrogen peroxide.

The results presented in Table 9 can also be viewed as the difference in log reduction between the hypochlorite alone controls and the binary system treatments. These data are collected in Table 10, shown below.

TABLE 10
A
Log Reduction Between Hypochlorite Alone and Binary
System Treatments Against Bacillus subtilis Spores
with Non-Acidified Peroxide

| Log Reduction with | | Non Acidified Peroxide mM | | | |
|---|---|---|---|---|---|
| Non-Acidified Peroxide | | 0.00 | 35.25 | 3.53 | 0.35 |
| NaOCl mM | 7.05 | 4.80 | 6.05 | | |
| | 0.705 | 2.59 | 3.23 | 2.53 | |
| | 0.0705 | -0.17 | 0.11 | | -0.14 |

B
Log Reduction Between Hypochlorite Alone and Binary
System Treatments Against Bacillus subtilis Spores
with Acidified Peroxide

| Log Reduction with | | Acidified Peroxide mM | | | |
|---|---|---|---|---|---|
| Acidified Peroxide | | 0.00 | 35.25 | 3.53 | 0.35 |
| NaOCl mM | 7.05 | 4.80 | 5.23 | | |
| | 0.705 | 2.59 | 3.02 | 2.62 | |
| | 0.0705 | -0.17 | -0.02 | | -0.11 |

C
Differences in Log Reduction Between Hypochlorite Alone
and Binary System Treatments Against Bacillus subtilis Spores
with Non-Acidified Peroxide

| Log Redn Difference | | Non Acidified Peroxide | | | |
|---|---|---|---|---|---|
| with Non-Acidified | | mM | | | |
| Peroxide | | 0.00 | 35.25 | 3.53 | 0.35 |
| NaOCl mM | 7.05 | — | 1.25 | | |
| | 0.705 | — | 0.64 | 0.06 | |
| | 0.0705 | — | 0.28 | | 0.03 |

D
Differences in Log Reduction Between Hypochlorite Alone
and Binary System Treatments Against Bacillus subtilis Spores
with Acidified Peroxide

| Log Redn Difference | | Acidified Peroxide mM | | | |
|---|---|---|---|---|---|
| with Acidified Peroxide | | 0.00 | 35.25 | 3.53 | 0.35 |
| NaOCl mM | 7.05 | — | 0.43 | | |
| | 0.705 | — | 0.43 | 0.03 | |
| | 0.0705 | — | 0.15 | | 0.06 |

The data support the conclusion that both hypochlorite followed by added peroxide and hypochlorite followed by added acidified peroxide give superior kill when compared to that of hypochlorite alone. Specifically, the use of the binary system with non-acidified peroxide gave 1.25 log increase (18-fold increase) in kill, and the use of acidified peroxide gave up to 0.43 log increase (3-fold increase) in kill when compared to equivalent levels of hypochlorite alone. A higher concentration of hydrogen peroxide also appears to enhance the effect of the binary system.

Since the data shown in Table 9 suggests that higher concentrations of hydrogen peroxide increase the microbicidal activity of the binary system, even at sub-microbicidal peroxide concentrations, the effect of increasing its concentration from 35 mM to 88 mM in the binary system was tested on Bacillus subtilis spores. The results are shown in Table 11.

TABLE 11
Microbicidal Activity of the Binary System Against Bacillus subtilis Spores

| NaOCl | NaOCl | H₂O₂ | H₂O₂ | Starting | Total | Log 10 | |
|---|---|---|---|---|---|---|---|
| Conc | time | Conc | Time | Inoculum | Viability | (CFU + 1) | Log |
| mM | (min) | mM | (min) | (CFU) | (CFU) | Survivors | Reduction |
| 7.0 | 60 | 88 | 30 | 1500000 | 7 | 0.9 | 5.3 |
| 3.5 | 60 | 88 | 30 | 1500000 | 102 | 2.0 | 4.2 |
| 0.7 | 60 | 88 | 30 | 1500000 | 1316 | 3.1 | 3.1 |
| 7.0 | 60 | | | 1500000 | 20 | 1.3 | 4.9 |
| 3.5 | 60 | | | 1500000 | 546 | 2.7 | 3.4 |
| 0.7 | 60 | | | 1500000 | 3304 | 3.5 | 2.7 |

Note:
The starting inoculum was 6.2 log 10.
The volumes of sodium hypochlorite, and hydrogen peroxide, were 0.5 ml each.

These data show no improvement over the data presented in Table 9 and Table 10. The difference in log reduction between the hypochlorite control alone and the binary system treatment is presented in Table 12 below.

TABLE 12
A
Log Reduction Between Hypochlorite Alone
and Binary System Treatments Against Bacillus subtilis Spores

| | | Non | |
|---|---|---|---|
| | | Acidified | |
| Log Reduction | | Peroxide | |
| with Non- | | mM | |
| Acidified Peroxide | | 0.00 | 88.20 |
| NaOCl mM | 7.00 | 4.87 | 5.28 |
| | 3.50 | 3.44 | 4.17 |
| | 0.70 | 2.66 | 3.06 |

B
Differences in Log Reduction Between Hypochlorite Alone
and Binary System Treatments Against Bacillus subtilis Spores

| | | Non | |
|---|---|---|---|
| Log Redn | | Acidified | |
| Difference with | | Peroxide | |
| Non-Acidified | | mM | |
| Peroxide | | 0.00 | 88.20 |
| NaOCl mM | 7.00 | — | 0.41 |
| | 3.50 | — | 0.72 |
| | 0.70 | — | 0.40 |

These data suggest that an increase in peroxide concentration from 35 mM to 88 mM provides no improvement in kill to the binary system. This is in agreement with the observations reported in Example 1 where time proved to be the most important influence on microbicidal activity.

### Example 4

### Microbicidal Activity of the Binary System Against Staphylococcus aureus at Reduced Exposure Time to Hydrogen Peroxide

The microbicidal activity of the binary system against Staphylococcus aureus was further investigated. Shorter hydrogen peroxide treatment times in step 2 of the binary system were tested to determine the effect of time on the reaction between organisms chlorinated in step 1 and hydrogen peroxide. Table 13 below presents the results obtained after execution of the binary system protocol on Staphylococcus aureus.

TABLE 13
Microbicidal Activity of the Binary System Against Staphylococcus aureus

| NaOCl | NaOCl | H₂O₂ | H₂O₂ | Starting | Total | Log 10 | |
|---|---|---|---|---|---|---|---|
| Conc | time | Conc | Time | Inoculum | Viability | (CFU + 1) | Log |
| mM | (min) | mM | (min) | (CFU) | (CFU) | Survivors | Reduction |
| 0.05 | 15 | 88 | 5 | 1390000 | 118 | 2.1 | 4.1 |
| 0.05 | 15 | 88 | 15 | 1390000 | 10 | 1.0 | 5.1 |
| 0.04 | 15 | 88 | 5 | 1390000 | 143 | 2.2 | 4.0 |
| 0.04 | 15 | 88 | 15 | 1390000 | 116 | 2.1 | 4.1 |
| 0.03 | 15 | 88 | 5 | 1390000 | 5880 | 3.8 | 2.4 |
| 0.03 | 15 | 88 | 15 | 1390000 | 2044 | 3.3 | 2.8 |
| 0.02 | 15 | 88 | 5 | 1390000 | 1162000 | 6.1 | 0.1 |
| 0.02 | 15 | 88 | 15 | 1390000 | 1274 | 3.1 | 3.0 |

Note:
The starting inoculum was 6.1 log₁₀ for S. aureus.
The volumes of sodium hypochlorite and peroxide were 0.5 ml each.
Chlorhexidine = Chlorhexidine Gluconate;
IPA = Isopropyl Alcohol;
PVI = Povidone Iodine

At all hypochlorite concentrations tested, increasing hydrogen peroxide treatment time from 5 to 15 minutes improved the performance of the binary system up to 3 log10 CFU. This increase in kill is not attributable to hydrogen peroxide alone. As shown in Table 1, 88 mM of hydrogen peroxide is not microbicidal to S. aureus for up to 30 minutes. These data suggest that the chlorinated intermediates formed during the first phase of the binary system require more time to react with hydrogen peroxide than does sodium hypochlorite for which the reaction is diffusion limited.

### Example 5

### Total Kill of Staphylococcus aureus and Bacillus subtilis Spores by the Binary System

The following table shows the microbicidal activity obtained using the binary system against approximately 6 log10 inoculum of Staphylococcus aureus, or Bacillus subtilis spores.

TABLE 14
Total Kill of Staphylococcus aureus and Bacillus subtilis Spores by the Binary System

| | NaOCl | NaOCl | H₂O₂ | H₂O₂ | Starting | Total | Log 10 | |
|---|---|---|---|---|---|---|---|---|
| Organism | Conc | time | Conc | Time | Inoculum | Viability | (CFU + 1) | Log |
| name | mM | (min) | mM | (min) | (CFU) | (CFU) | Survivors | Reduction |
| S. aureus | 0.07 | 15 | 0.35 | 30 | 2530000 | 0 | 0.0 | 6.4 |
| S. aureus | 0.06 | 15 | 0.3 | 30 | 2530000 | 0 | 0.0 | 6.4 |
| B. subtilis | 7.05 | 60 | 35.25 | 30 | 1120000 | 0 | 0.0 | 6.1 |

Note:
The starting inoculum was 6.4 log10 for S. aureus, and 6.1 log10 for B. subtilis.
The volumes of sodium hypochlorite and peroxide were 0.5 ml each.

The solutions obtained at the completion of treatment with the binary system do not contain residual hypochlorite. The excess hydrogen peroxide introduced in the second step of the binary system, although not microbicidal on its own reacts with the hypochlorite remaining at the end of step 1 to form singlet oxygen. This results in complete kill and a non-toxic, hypochlorite free solution containing small amounts of hydrogen peroxide and sodium chloride.

### Example 6

### Microbicidal Activity of Commercially Available Antiseptics Against Staphylococcus aureus and Bacillus subtilis Spores

The performance of commonly used antiseptics was tested against Bacillus subtilis and Staphylococcus aureus, following the general procedure of Example 1, to assess the improvement provided by the binary system over these compounds.

After 60 minutes of exposure to the organisms, the individual antiseptics, indicated in Table 15 and Table 16, were neutralized using the corresponding neutralizing solutions prepared as listed below in Materials. Chlorhexidine and IPA were neutralized with 500 microliters of a solution containing 3% Saponin, 3% Tween-80, 0.3% lecithin, and 0.1% histidine. Povidone Iodine was neutralized with 500 microliters of thiosulfate solution (2.4%). The results are presented in Table 15 and Table 16.

Chlorhexidine Gluconate (Spectrum, Cat # CH126) was diluted in sterile water to prepare the Chlorhexidine solutions at the desired concentrations.

Isopropyl Alcohol 70% (Spectrum, Cat # IS120) was diluted in sterile water to prepare concentrations required for this study.

Povidone Iodine USP (Spectrum, Cat # P 0330) was used as a 10% solution (containing 1% titratable iodine).

1-Histidine (Spectrum, Cat # H1021) was used as a 0.1% solution.

Saponin (Spectrum, Cat # S1022) was used as a 3% solution.

Lecithin (Spectrum, Cat # L1083) was used as a 0.3% solution.

Tween 80 (Fisher, Cat # T164-500) was used as a 3% solution.

TABLE 15
Microbicidal Activity of Commonly Used Antiseptics Against Bacillus subtilis Spores

| | Chlorhexidine | IPA | IPA | PVI | PVI | Starting | Total | Log 10 | |
|---|---|---|---|---|---|---|---|---|---|
| Chlorhexidine | Time | Conc | Time | Conc | Time | Inoculum | Viability | (CFU + 1) | Log |
| Conc (%) | (min) | (%) | (min) | (%) | (min) | (CFU) | (CFU) | Survivors | Reduction |
| 20 | 60 | | | | | 1865000 | 350000 | 5.5 | 0.7 |
| 2 | 60 | | | | | 1865000 | 102200 | 5.0 | 1.3 |
| 0.200 | 60 | | | | | 1865000 | 20020 | 4.3 | 2.0 |
| 0.020 | 60 | | | | | 1865000 | 672000 | 5.8 | 0.4 |
| | | 70 | 60 | | | 1865000 | 2044000 | 6.3 | 0.0 |
| | | 7 | 60 | | | 1865000 | 2352000 | 6.4 | −0.1 |
| | | 0.70 | 60 | | | 1865000 | 2170000 | 6.3 | −0.1 |
| | | 0.07 | 60 | | | 1865000 | 2422000 | 6.4 | −0.1 |
| | | | | 10.00 | 60 | 1120000 | 189000 | 5.3 | 0.8 |

Note:
The starting inoculum for B. subtilis was 6.3 log10 for Chlorhexidine and IPA, and 6.1 log10 for PVI.
The volumes of Chlorhexidine, IPA, and PVI were 0.5 ml each.
Chlorhexidine = Chlorhexidine Gluconate;
IPA = Isopropyl Alcohol;
PVI = Povidone Iodine

Isopropyl alcohol did not demonstrate activity against B. subtilis spores at any concentration tested. Chlorhexidine provided some decrease in organism count, however the concentration commonly used (2%) yielded only 1.3 log10 reduction within 60 minutes. Hypochlorite alone is clearly the most effective single action antimicrobial against spores even at concentrations as low as 7 mM, which is 1/100th that of undiluted liquid bleach (see Table 8).

TABLE 16
Microbicidal Activity of Commonly Used Antiseptics Against Staphylococcus Aureus

| | | IPA | IPA | PVI | PVI | Starting | Total | Log 10 | |
|---|---|---|---|---|---|---|---|---|---|
| Chlorhexidine | Chlorhexidine | Conc | Time | Conc | Time | Inoculum | Viability | (CFU + 1) | Log |
| Conc (%) | Time (min) | (%) | (min) | (%) | (min) | (CFU) | (CFU) | Survivors | Reduction |
| 20 | 15 | | | | | 1475000 | 0 | 0.0 | 6.2 |
| 2 | 15 | | | | | 1475000 | 0 | 0.0 | 6.2 |
| 0.200 | 15 | | | | | 1475000 | 0 | 0.0 | 6.2 |
| 0.020 | 15 | | | | | 1475000 | 0 | 0.0 | 6.2 |
| | | 70 | 15 | | | 1475000 | 0 | 0.0 | 6.2 |
| | | 7 | 15 | | | 1475000 | 1834000 | 6.3 | −0.1 |
| | | 0.70 | 15 | | | 1475000 | 1988000 | 6.3 | −0.1 |
| | | 0.07 | 15 | | | 1475000 | 1932000 | 6.3 | −0.1 |
| | | | | 10.00 | 15 | 1475000 | 0 | 0.0 | 6.2 |

Note:
The starting inoculum was 6.2 log10.
The volumes of Chlorhexidine, IPA, and PVI were 0.5 ml each.
Chlorhexidine = Chlorhexidine Gluconate;
IPA = Isopropyl Alcohol;
PVI = Povidone Iodine

The data presented in Tables 1, 7, and 12 illustrate the potent and well established microbicidal action of hypochlorite alone. The experimental Examples 1-7 demonstrate that Phase 2 exposure to hydrogen peroxide augments the microbicidal action of Phase 1 hypochlorite. However, the crucial significance of Phase 2 of the binary system is that it provides an essential mechanism for controlling hypochlorite activity and yields innocuous dilute saline solution.

### Example 7

### Addition of Surface Active Agents

To illustrate the foaming effect following addition of peroxide to hypochlorite, six surface active agents were individually mixed with 5.25% bleach in order to achieve a concentration of 2% v/v. Two ml of each of these solutions were added to separate graduated polypropylene centrifuge tubes, followed in each case by 2 ml of 30% hydrogen peroxide. The volume of foam obtained was recorded immediately (30 sec) after addition of hydrogen peroxide and 5 minutes after addition of hydrogen peroxide. The results for the different agents tested are shown in the Table 17 below and illustrated by pictures taken immediately after mixing of the bleach and the hydrogen peroxide as well as 5 minutes later.

TABLE 17
Foam Experiments

| | | | | Sodium | Sodium | | | |
|---|---|---|---|---|---|---|---|---|
| | | Polyoxy- | | octylphen- | octylphen- | | | |
| | | ethylene | Polyoxyl 3 | oxypolyeth- | oxypolyeth- | | | Polyoxy- |
| | | sorbitan | 5-castor oil | oxyethyl | oxyethyl | Sodium | | ethylene |
| | | monooleate | (Cremophor | sulfonate | sulfonate | dodecyl | | lauryl ether |
| Time after | | (Tween 80) | EL) | (Triton | (Triton | sulfate | Benzalkonium | (BRIJ 35) |
| mixing | | 2% | 2% | x200) 2% | x200) 1% | (SDS) 2% | Chloride 2% | 2% |
| 0-30 | sec | 30 ml | 30 ml | 32.5 ml | 32.5 ml | 32.5 ml | 37.5 ml | 30 ml |
| 5 | min | 35 ml | 30 ml | 27.5 ml | 27.5 ml | 35 ml | 5 ml | 35 ml |

The characteristics of the foam generated with 2% Tween 80, 2% Cremophor EL, 2% BRIJ 35, and 2% SDS were similar in volume, consistency and duration. 2% Triton X-200- and 1% Triton X-200 were similar, but with somewhat diminished foam duration. The foam generated by 2% Benzalkonium Chloride was loose and ephemeral.

### Example 8

### Sodium Hypochlorite and Hydrogen Peroxide UV Spectra

UV spectra were determined on a GBC UV/VIS spectrophotometer, Model 918. Spectra were rendered using GBC Spectral Software. Solutions of sodium hypochlorite and hydrogen peroxide were prepared by dilution of stock with distilled water. Individual spectra were taken at room temperature in 1 cm path-length quartz cuvettes using water as the reference.

Diluted solutions of sodium hypochlorite were prepared as follows: Stock bleach at 5.25% is 705 mM sodium hypochlorite.

To prepare 20 ml of a 2 mM solution, 60 μL of the stock solution was added to 19,940 μL of distilled water.

To prepare 20 ml of 0.1 mM sodium hypochlorite, 1 ml of the 2 mM hypochlorite solution, prepared as per above, was added to 19 ml of distilled water.

Dilute solutions of hydrogen peroxide were prepared as follows: Stock hydrogen peroxide is 8821 mM.

To prepare 20 ml of a 705 mM solution of hydrogen peroxide, 1,599 μL of the stock solution was added to 18,401 μL of distilled water.

To prepare 20 ml of a 10 mM peroxide solution, 300 μL of a 705 mM solution of hydrogen peroxide was added to 19,700 μL of distilled water.

To prepare 20 ml of a 2 mM solution, 60 μL of the 705 mM solution was added to 19,940 μL of distilled water.

To prepare 20 ml of 0.5 mM hydrogen peroxide, 5 ml of the 2 mM peroxide solution, prepared as per above, was added to 15 ml of distilled water.

To prepare 20 ml of 0.1 mM hydrogen peroxide, 1 ml of the 2 mM peroxide solution, prepared as per above, was added to 19 ml of distilled water.

Spectra of dilutions of hydrogen peroxide alone as well as bleach alone were determined in order to ascertain the concentration that resulted in an absorbance maximum of 0.5 to 1 absorbance unites (AU). The spectra of equimolar mixtures of peroxide and hypochlorite as well as 5 fold excess hydrogen peroxide were determined immediately after mixing and 5 minutes later.

Spectra are provided for the following 12 experimental solutions:

1. 35 mM H₂O₂ vs. water

2. 7 mM H₂O₂ vs. water

3. 705 mM NaOCl vs. water

4. 7.05 mM NaOCl vs. water

5. 2 mM NaOCl vs. water

6. 0.1 mM NaOCl vs. water

7. 10 mM H₂O₂ vs. water

8. 2 mM H₂O₂ vs. water

9. 2 mM NaOCl+2 mM H₂O₂ at 0 minutes vs. water

10. 2 mM NaOCl+2 mM H₂O₂ at 5 minutes vs. water

11. 2 mM NaOCl+10 mM H₂O₂ at 0 minutes vs. water

12. 2 mM NaOCl+10 mM H₂O₂ at 5 minutes vs. water

The pH of the binary system mixtures was also determined and is presented in the table below.

TABLE 18
pH Of Binary System Mixtures

| NaOCl | H₂O₂ | pH at 1 minute | pH at 5 minutes |
|---|---|---|---|
| 2 mM | 2 mM | 8.39 | 7.36 |
| 2 mM | 10 mM | 7.59 | 7.23 |

Hydrogen peroxide at 1 and 5 equivalents completely remove all evidence of hypochlorite at 2 mM concentration.

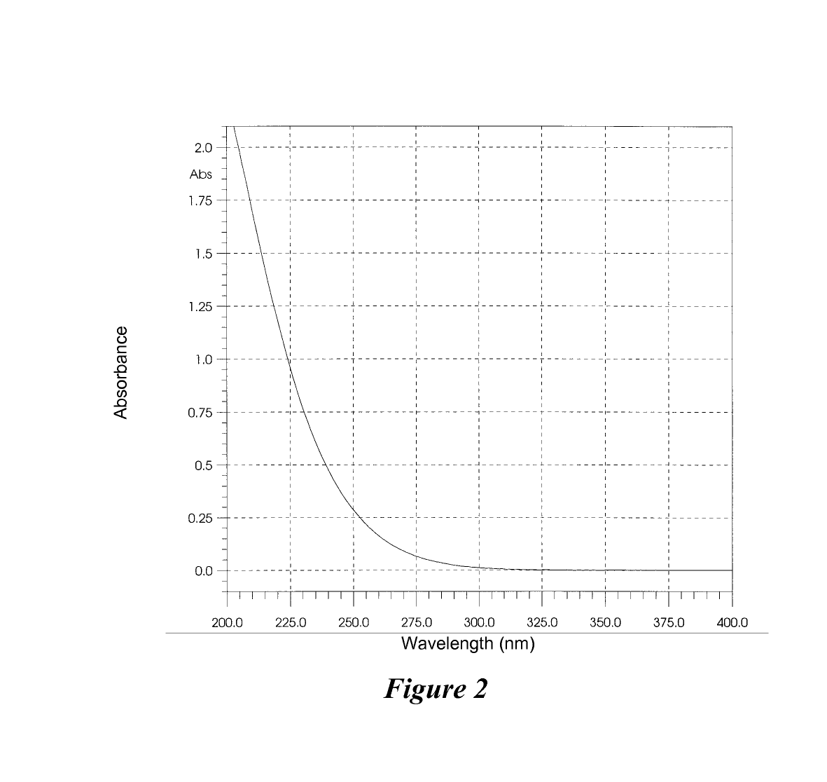

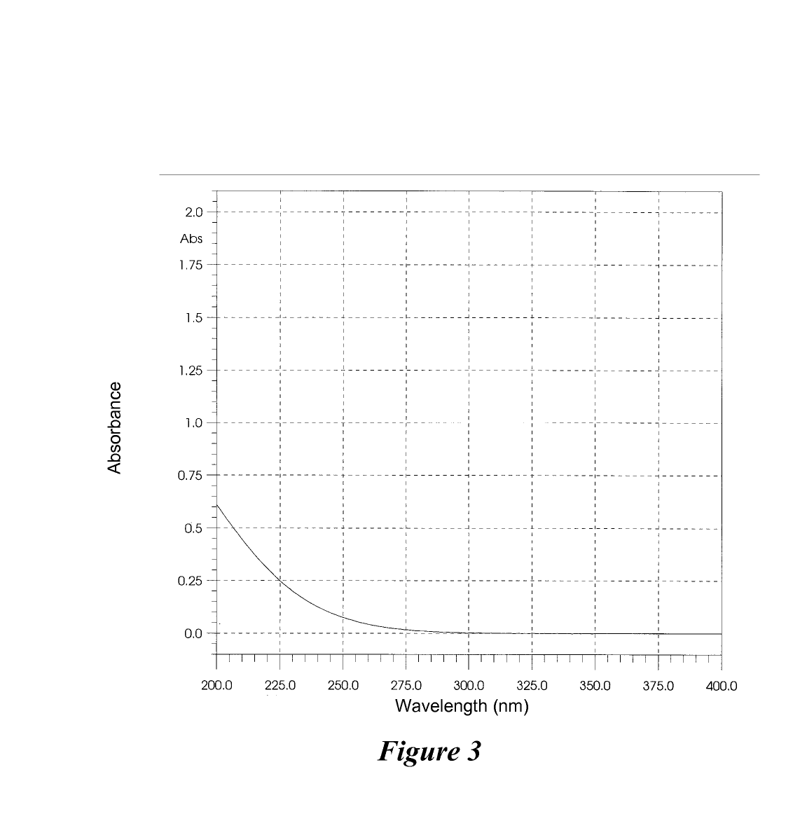

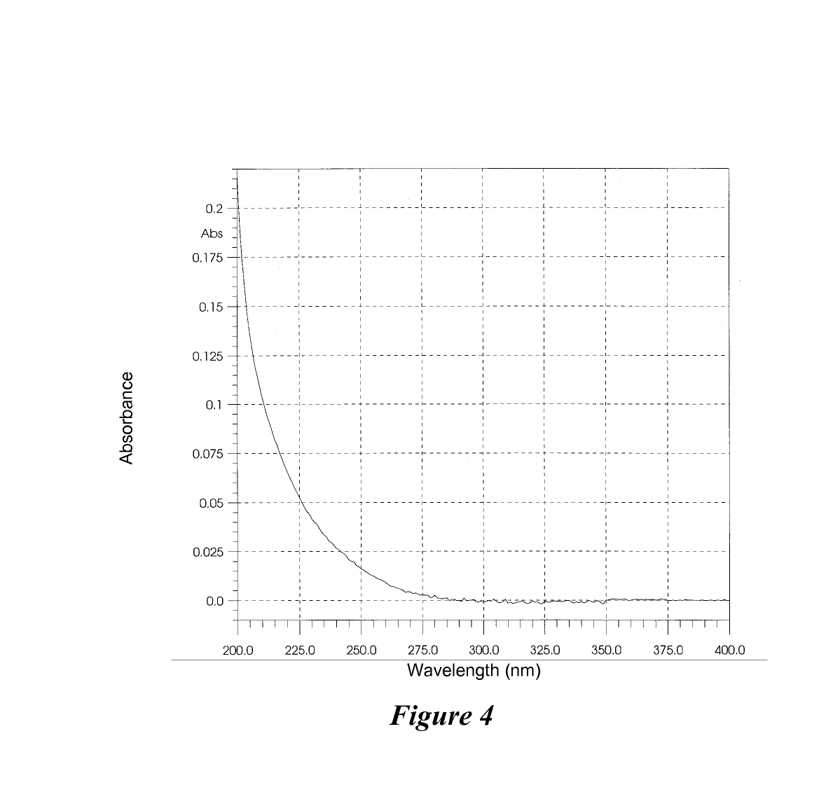

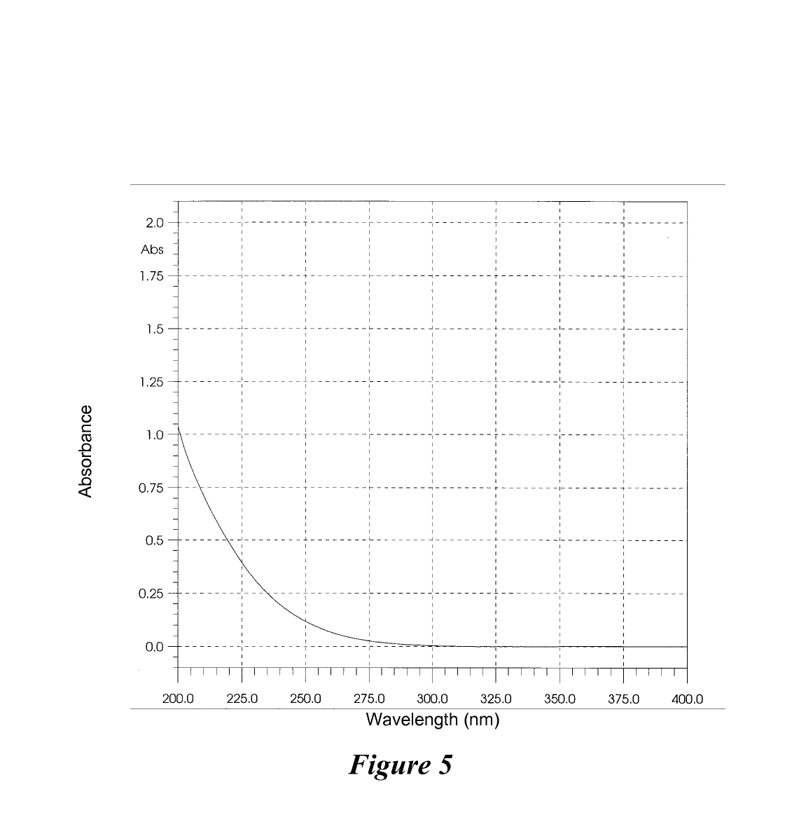

FIG. 1 illustrates the UV spectra of a solution of sodium hypochlorite at 2 mM concentration. FIG. 2 illustrates the UV spectra of a 10 mM solution of hydrogen peroxide. FIG. 3 illustrates the UV spectra of a 2 mM solution of hydrogen peroxide. FIG. 4 illustrates the UV Spectra of an equimolar solution of sodium hypochlorite and hydrogen peroxide at 2 mM. The pH of the mixture at one minute after addition of the hydrogen peroxide to the sodium hypochlorite was 8.39. After 5 minutes the pH was 7.36. The resulting pH, overall reduction of absorbance, in general, and loss of absorbance between 250 nm and 350 nm, in particular, demonstrate that the reaction of hydrogen peroxide with sodium hypochlorite results in complete neutralization of the sodium hypochlorite and yields an innocuous dilute pH-neutral, saline solution as reaction product. FIG. 5 illustrates the UV spectra of a solution containing 2 mM sodium hypochlorite and 10 mM of hydrogen peroxide; a 5-fold molar excess of hydrogen peroxide. The pH of the mixture at one minute after addition of the hydrogen peroxide to the sodium hypochlorite was 7.59. After 5 minutes the pH was 7.23. The resulting drop in pH, reduction of absorbance at 200 nm and loss of absorbance between 250 nm and 350 nm, specific to sodium hypochlorite, demonstrate that the reaction of hydrogen peroxide with sodium hypochlorite results in complete neutralization of the sodium hypochlorite. The resulting dilute pH-neutral saline solution contains residual hydrogen peroxide as evidenced by the UV spectra.

### Example 9

### Sterilization of the Skin Surface

The following representative method relates to the preparation of a sterile field on the surface of skin prior to carrying out a procedure, e.g., collecting a blood culture.

Phase 1 (dehydrogenation/chlorination): A solution ranging in NaOCl concentration from about 0.3 to 2.0% (w/v) (i.e., 40 to 270 mM NaOCl) is sprayed or otherwise directly applied to the skin surface. The amount of Phase 1 solution should be sufficient to cover the area to be sterilized, but excess solution should be avoided. A 4×4 gauze pad is placed over the area to be sterilized and 0.6% NaOCl is applied directly to the gauze pad. The area is gently rubbed to facilitate contact and cleansing.

Phase 2 (singlet oxidation/oxygenation): After a relatively short contact period, e.g., about one minute, a solution of H₂O₂ ranging in concentration from about 0.2 to 1.0% (i.e., about 59 to about 294 mM H₂O₂) is sprayed or otherwise applied to the same skin surface. The amount of Phase 2 solution applied should be more than twice that of the Phase 1 solution to insure that any residual NaOCl is completely reacted with the H₂O₂ yielding 1O₂*.

The initiation of Phase 2 singlet oxidation phase, terminates the chlorination phase, converting the remaining NaOCl to saline and 1O₂*. The soapy alkaline character of NaOCl in Phase 1 is converted to essential neutral aqueous character. The alkaline 0.6% NaOCl solution is neutralized to about two-thirds normal saline (˜0.6% NaCl).

Any chloramines produced during the chlorination phase will also be converted to 1O₂*. If necessary, this later conversion can be facilitated by mild acidification of the Phase 2H₂O₂ solution used to initiate the singlet oxidation phase of reaction.

The keratinized epithelium of intact skin provides protection during the relatively short exposure to NaOCl. If necessary, the potency of the preparation can be adjusted by changing the NaOCl concentration or exposure duration. Even at significantly lower concentrations, NaOCl is expected to exert much greater antiseptic action than povidone-iodine or chlorhexidine.

### Example 10

### Sterilization of Surfaces and Biofilms

The following representative method relates to the killing of microbes on surfaces and the oxidative removal of biofilms. The following applications are directed to sterilization of surfaces, e.g., medical and/or scientific instruments sterilization, countertop sterilization and the like.

The efficacy of NaOCl as an agent for surface sterilization and removal of biofilms is well established. Its use as described should be limited to relatively inert surfaces. In addition to killing all surface-associated microbes, the combined chlorination and singlet oxidation will effectively oxygenate (combust) protein and organic material adhering to the surface. As such, the binary system as described is not advised for direct use on biological materials such as tooth surfaces.

Phase 1 (dehydrogenation/chlorination). A solution ranging from 3 to 10% (w/v) of NaOCl (i.e., 0.4 to 1.3 M NaOCl) is sprayed or otherwise directly applied to the surface to be sterilized.

Phase 2 (singlet oxidation/oxygenation). After an adequate contact period, ranging from 1 to 30 minutes, the Phase 2H₂O₂ solution equivalent or greater than the hypochlorite solution of Phase 1, ranging in concentration from 1 to 10% (0.3 to 2.9 M H₂O₂), is sprayed or otherwise applied to the same surface. The residual NaOCl will react with the H₂O₂ liberating copious amounts of 1O₂* bubbles.

If no bubbles are generated, then insufficient NaOCl was employed in the Phase 1 reaction, and as such, the sterilization should be repeated using the same sequence of reagent steps. Phase 2 addition of H₂O₂ should continue until bubble generation ceases, indicating the exhaustion of residual NaOCl. If the presence of a small concentration of residual H₂O₂ presents a problem, catalase can be added to remove this residual H₂O₂.

### Example 11

### Field Preparation of Potable Water

The following example illustrates the killing of microbes and oxygenation of organic material in water so as to render it potable for human use.

Phase 1 (dehydrogenation/chlorination). Before sterilization, the water is coarsely filtered, if necessary, to remove excess organic material. Once reasonably clarified, the raw water is subjected to Phase 1 treatment with concentrated NaOCl, such as ranging from 6 to 30% (0.8 to 4.0 M NaOCl). For example, if the water to be treated is relatively clean, a small quantity of hypochlorite can be used, e.g., 3 ml (a teaspoon) of 30% NaOCl added to a liter of raw water would yield a 0.09% NaOCl, i.e., a one-tenth normal saline solution. However, if necessary NaOCl or Ca(OCl)₂ can be greatly increased (at least up to 0.5% volume hypochlorite per volume of water to be treated). The Phase 1 solution should be well mixed and allowed to sit for at least thirty minutes.

Phase 2 (singlet oxidation/oxygenation). After an adequate contact period, such as 30 minutes, a small amount (0.5 ml; 10 drops) of 10% H₂O₂ solution (2.9 M H₂O₂) is added to the Phase 1 treated raw water. The final molar quantity of peroxide added should be equivalent to the quantity of hypochlorite added in Phase 1. Any residual NaOCl in the Phase 1-treated water reacts with the H₂O₂ to liberate 1O₂* bubbles. The release of 1O₂* bubbles on addition of Phase 2H₂O₂ should be observed.

The absence of bubbles on addition of H₂O₂ may indicate that insufficient NaOCl was employed in the Phase 1 reaction, and as such, the sterilization cycle should be repeated using the same sequence of reagent steps.

When bubbles are noted at initiation of Phase 2, H₂O₂ addition is continued with vigorous shaking until bubble generation ceases, indicating exhaustion of residual NaOCl. The total molar quantity of peroxide should be roughly equivalent to the molar quantity of hypochlorite added in Phase 1. The small concentration of residual H₂O₂ remaining at the end of Phase 2 treatment can be removed by adding a small amount of catalase to the Phase 2 treated water.

While the preferred embodiment of the invention has been illustrated and described, it will be appreciated that various changes can be made therein without departing from the spirit and scope of the invention.

All references, including patents or applications cited herein, are incorporated by reference in their entirety, as if written herein.

The embodiments of the invention in which an exclusive property or privilege is claimed are defined as follows:

1. A method of decontaminating a surface or liquid target comprising contacting the target with a first composition comprising hypohalite for a first treatment time, and then contacting the target with a second composition comprising a sufficient amount of peroxide to react with substantially all of the hypohalite in the first composition for a second treatment time.

2. The method of claim 1 wherein the molar ratio of hypohalite in the first composition to peroxide in the second composition is 1:1 or less.

3. The method of claim 2 wherein the molar ratio of hypohalite in the first composition to peroxide in the second composition is 1:2 or less.

4. The method of claim 3 wherein the molar ratio of hypohalite in the first composition to peroxide in the second composition is 1:4 or less.

5. The method of claim 1 wherein the first hypohalite composition comprises an aqueous solution of hypohalite.

6. The method of claim 5 wherein the hypohalite in the first composition is an alkali metal hypohalite.

7. The method of claim 6 wherein the alkali metal hypohalite is sodium hypochlorite.

8. The method of claim 7 wherein the concentration of sodium hypochlorite is from about 0.0001 mM to about 5 M.

9. The method of claim 7 wherein the concentration of sodium hypochlorite is from about 0.001 mM to about 1 M.

10. The method of claim 7 wherein the concentration of sodium hypochlorite is from about 0.01 mM to about 700 mM.

11. The method of claim 1 wherein the second peroxide composition comprises an aqueous solution of peroxide.

12. The method of claim 11 wherein the peroxide in the second composition is an alkali metal peroxide.

13. The method of claim 12 wherein the alkali metal peroxide is sodium peroxide.

14. The method of claim 11 wherein the peroxide in the second composition is hydrogen peroxide.

15. The method of claim 14 wherein the concentration of hydrogen peroxide is from about 0.001 mM to about 10 M.

16. The method of claim 14 wherein the concentration of hydrogen peroxide is from about 0.01 mM to about 1 M.

17. The method of claim 14 wherein the concentration of hydrogen peroxide is from about 0.1 mM to about 880 mM.

18. The method of claim 1 where the hypohalite in the first composition is sodium hypochlorite and the peroxide in the second composition is hydrogen peroxide.

19. The method of claim 18 wherein the concentration of sodium hypochlorite is from about 0.0001 mM to about 5 M and the concentration of hydrogen peroxide is from about 0.001 mM to about 10 M.

20. The method of claim 18 wherein the concentration of sodium hypochlorite is from about 0.001 mM to about 1 M and the concentration of hydrogen peroxide is from about 0.01 mM to about 1 M.

21. The method of claim 1 wherein the first treatment time is at least 1 minute.

22. The method of claim 1 wherein the first treatment time is at least 5 minutes.

23. The method of claim 1 wherein the first treatment time is at least 10 minutes.

24. The method of claim 1 wherein the second treatment time is at least 5 seconds.

25. The method of claim 1 wherein the second treatment time is at least 30 seconds.

26. The method of claim 1 wherein the second treatment time is at least 30 minutes.

27. The method of claim 1 where the surface is contaminated with a pathogenic agent.

28. The method of claim 27 wherein the pathogenic agent is selected from the group consisting of a bacterium, fungi, yeast, virus, and prions.

29. The method of claim 1 wherein the pathogenic agent is a microorganism in vegetative or spore form.

30. The method of claim 29 wherein the microorganism is in spore form.

31. The method of claim 30 wherein the microorganism in spore form is selected from the group consisting of Bacillus, Clostridia, and Sporosarcina.

32. The method of claim 31 wherein the microorganism in spore form is selected from the group consisting of Bacillus anthracis, Bacillus subtilis, Bacillus thuringiensis, and Clostridia botulinum.

33. The method of claim 1 wherein the target is an animal.

34. The method of claim 33 wherein the animal is human.

35. The method of claim 34 wherein the surface target is skin or hair.

36. The method of claim 1 wherein the surface target is on an inanimate object.

37. The method of claim 1 wherein the liquid target is contaminated water.

38. The method of claim 1 wherein the first composition, the second composition, or both the first and second compositions, further comprise one or more surfactants, detergents, or co-solvents.

39. The method of claim 38 wherein the surfactant or detergent is selected from group consisting of non-ionic, anionic, cationic zwitter-ionic surfactants, and detergents.

40. The method of claim 39 wherein the surfactant is selected from the group consisting of polyoxyethylene sorbitan esters, polyoxyethylene ethers, alkyl polyglucosides, alcohol or phenol ethoxylates, alkylamine ethoxylates, alkylarylether sulfates or sulfonates, alkyldiphenyloxide disulfonates, and alkylarylammonium halides.

41. The method of claim 40 wherein the surfactant is selected from the group consisting of polyoxyethylene sorbitan monooleate, polyethoxy cetylether, sodium octylphenoxypolyethoxyethyl sulfonate, sodium dodecyl sulfate, sodium deoxycholate, benzalkonium chloride, dodecyltrimethylammonium bromide, polyoxyl castor oil, polyoxyl hydrogenated castor oil, polyethylene-polypropylene glycol, octyl-beta-D-glucopyranoside, triethyleneglycol monododecylether, and dimethylpalmitylammonio-propane sulfonate.

42. The method of claim 38 wherein the co-solvent is selected from the group consisting of alcohols, glycerols, and glycols.

43. The method of claim 42 wherein the co-solvent is selected from the group consisting of isopropyl alcohol, butanol, glycerin, propylene glycol, and butanediols.

44. The method of claim 1 wherein the first composition, the second composition, or both the first and second compositions, further comprise one or more gelling agents, thixotropic agents, or viscosity enhancing agents.

45. The method of claim 44 wherein the gelling agent, thixotropic agent or viscosity enhancing agent is selected from the group consisting of amorphous colloidal silica gel, polyethylene glycols, methoxypolyethylene glycols, ethylcellulose, methylcellulose, hydroxypropylcellulose, hydroxypropylmethylcellulose, and hydroxyethylcellulose, gelatin, and alginates.

46. The method of claim 1 wherein the first composition, the second composition, or both the first and second compositions, further comprise one or more detection agents.

47. The method of claim 46 wherein said first composition comprises a first detection agent and said second composition comprises a second detection agent wherein the first and second detection agents are not the same.

48. The method of claim 47 wherein the detection agent is selected from the group consisting of paint and dye.

49. The method of claim 48 wherein the detection agent is paint.

50. A kit for decontaminating a surface or a liquid target comprising a first container containing a first composition comprising hypohalite and a second container containing a second composition that comprises peroxide.

51. The kit of claim 50 wherein said first composition, said second composition, or both first and second compositions, further comprise one or more surfactants, detergents, co-solvents, gelling agents, thixotropic agents, viscosity enhancing agents, or detection agents.

52. The kit of claim 51 wherein said first composition comprises a first detection agent and said second composition comprises a second detection agent wherein the first and second detection agents are not the same.

53. The kit of claim 50 wherein the hypohalite is hypochlorite and the peroxide is hydrogen peroxide.
